(19) United States
(12) Patent Application Publication
(10) Pub. No.: US 2003/0139333 A1
(43) Pub. Date: Jul. 24, 2003

(54) Methods and compositions for promoting angiogenesis

(75) Inventors: Robert Pawliuk, Medford, MA (US); Phillipe LeBoulch, Charlestown, MA (US); Renhai Cao, Bromma (SE); Yihai Cao, Bromma (SE)

Correspondence Address: LAHIVE & COCKFIELD, 28 STATE STREET, BOSTON, MA, 02109, US

(73) Assignee: Genetix Pharmaceuticals, Inc., Cambridge, MA (US)

(21) Appl. No.: 10/198,917

(22) Filed: Jul. 19, 2002

(30) Foreign Application Priority Data
Jan. 18, 2002 (WO) PCT/US02/01666

(51) Int. Cl. A61K 48/00; A61K 38/18; A61K 9/70; A61F 13/00

(52) U.S. Cl. 514/12; 514/44; 424/93.2; 424/443

(57) ABSTRACT

Methods and compositions for promoting angiogenesis by delivering angiogenic factors are disclosed. Also disclosed are improved techniques for delivering angiogenic factors, for example, in the treatment of tissue ischemia.

This application claims priority to U.S. Provisional Patent Application Serial No. 60/264,457, filed on Jan. 26, 2001, and PCT Application No. PCT/US02/01666 filed Jan. 18, 2002, published in English in accordance with PCT Article 21(2), the contents of which are hereby incorporated herein.

### BACKGROUND OF THE INVENTION

The depletion of oxygen supply due to obstructed or inadequate blood supply is the common pathological state associated with various tissue ischemias, including myocardial ischemia, ischaemic bowel disease, and peripheral ischemia. The alleviation of tissue ischemia is critically dependent upon angiogenesis, the process by which new capillaries are generated from existing vasculature and tissue. The spontaneous growth of new blood vessels provide collateral circulation in and around an ischemic area, improves blood flow, and alleviates the symptoms caused by the ischemia. Although surgery or angioplasty may help to revascularize ischemic regions in some cases, the extent, complexity and location of the arterial lesions which cause the occlusion often prohibits such treatment.

Alternative methods for the treatment of chronic ischemia have focused on the delivery of angiogenic growth factors, over twenty of which are known. To date, modest but significant angiogenesis has been achieved following administration (e.g., by local injection) of exogenous factors to animal models. For example, purified recombinant VEGF-A has been demonstrated to elicit a modest but significant vascularization following injection into ischemic skeletal muscle tissue in a rabbit model of chronic limb ischemia [Takeshita et al., Circulation 90, 228:1994]. In addition, direct injection of vectors containing cDNA encoding VEGF-A has also been shown to induce a modest stimulation of angiogenesis in ischemia animal models in both skeletal and cardiac muscle [Takeshita et al., Biochemical and Biophysical Research Communications 227, 628:1996; and MacGovern et al., Human Gene Therapy 8, 215: 1997]. However, such local application often results in diffusion of the factors away from the desired site, thus diminishing angiogenic effect. Moreover, it is known that such bolus delivery of angiogenic factors generally results in a local and disorganized hodge-podge of new blood vessels, only a fraction of which (if any) contribute to amelioration of a blockage.

An additional limitation associated with current approaches for promoting angiogenesis and effective treatment of ischemia is the inability of stable blood vessels to form by application of single agents using known techniques. In past studies, when vessels were followed for several months after treatment of ischemic animal models, the vast majority of new vessels were observed to regress after the applied growth factor had become depleted. Thus, current approaches for ischemia therapy require repeated applications of factors to maintain newly formed vasculature.

Other related therapy methods attempt to circumvent the need for multiple applications by relying on the transplantation of autologous or non-autologous cells which can produce sustained levels of angiogenic proteins. In one such approach, a subject's endogenous cells are isolated, cultured, and transfected with expression vectors encoding angiogenic proteins. Following in vitro manipulations, these cells are injected back into the patient at the site of tissue ischemia. However, the drawbacks of this approach include the time and effort required to isolate, culture and transfect target cells from each individual patient, as well as difficulties in securing sustained expression of angiogenic proteins. In addition, sub-optimal cell survival and differentiation states of the cells following injection also limit the efficacy and of this approach.

To avoid these problems, cells have been obtained from non-patient (e.g., allogeneic), even non-human, sources and manipulated in the manner described above.

However, the modified non-patient or non-human cells are often rejected by the patient's own immune system, making this approach impractical too.

Accordingly, improved therapies for promoting tissue angiogenesis and generating stable vasculature in a safe, reliable, and non-invasive manner are needed to treat tissue ischemia and other related conditions.

### SUMMARY OF THE INVENTION

The present invention provides improved methods and compositions for promoting angiogenesis and/or cellular remodeling in selected areas of tissue.

Accordingly, the invention can be used to treat a variety of tissue ischemias, including, for example, myocardial ischemia, peripheral ischemia, pulmonary ischemia, limb ischemia, brain ischemia, retinal ischemia, nerve tissue ischemia, kidney ischemia, skin ischemia, subcutaneous tissue ischemia, ischemia of the gut and ischemia of the brain.

In one embodiment of the invention, angiogenic factors, including synergistic combinations thereof, are delivered to a localized area of ischemic tissue in an amount effective to treat the ischemia. The factors can be delivered in protein form or in the form of nucleic acids encoding the factors. Accordingly, the invention further includes improved methods and vehicles for delivering such angiogenic factors and/or combinations of factors, functional analogues of such factors, and nucleic acids encoding such factors.

In a particular embodiment, the present invention provides a method of treating ischemia by delivering a combination of PDGF-BB and bFGF to a selected area of tissue in an amount effective to treat the ischemia. As shown herein, delivery of PDGF-BB and bFGF promotes formation of vessels that remain stable even after the delivered factors have been depleted or are no longer present. Moreover, as part of the present invention, it has been unexpectedly discovered that PDGF-BB and bFGF act synergistically to promote angiogenesis and/or tissue remodeling in ischemic tissue. By this it is meant that the therapeutic effect of the combination is greater than that which would be expected from the effect of either factor alone. Accordingly, a broad variety of tissue ischemias can be treated by delivering PDGF-BB and bFGF, either alone or in further combination with another angiogenic factor, such as PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof.

In yet another embodiment, the present invention provides a method of promoting tissue remodeling by delivering one or more angiogenic factors to a selected area of tissue in an amount sufficient to cause remodeling of existing vasculature and/or cells. In a particular embodiment, the tissue is ischemic so that the delivered factor(s) serve to promote both restructuring of existing tissue and/or vessels, as well as growth of new vessels (i.e., angiogenesis), thereby treating the ischemia.

Therapeutic angiogenic factors, nucleic acids (e.g., genes) encoding the factors, and combinations thereof, such as PDGF-BB and bFGF or genes encoding PDGF-BB and bFGF, can be delivered to ischemic tissues using a variety of art recognized techniques. For example, they can be delivered by direct injection using any suitable injection system, such as a NOGA delivery system. They can also be delivered in a sustained-release formulation to control and sustain their delivery rate. They can also be delivered in the form of a functional analogue, such as an anti-idiotypic antibody to PDGF-BB and/or bFGF.

Accordingly, in further embodiments, the present invention provides a variety of improved techniques for delivering angiogenic factors and/or genes encoding the factors to selected areas of tissue. In one such embodiment, the factors (e.g., PDGF-BB and bFGF) are delivered in association with a polymer which controls release of the factors or genes onto or into a selected area of tissue. The polymer can comprise, for example, a matrix which consists of, e.g., heparin sepharose/alginate, chitosan/tricalcium phosphate sponge, poly-lactide-glycolide sponge, polylactide glycolic mesh, methyl cellulose, polysulfone, extrudable ethylene vinyl acetate, alginate/poly-L-lysine/alginate and agarose/poly-L-lysine/alginate

In yet another embodiment, the present invention provides a method for promoting angiogenesis by delivering angiogenic factors, such as those described above (e.g., PDFG-BB and bFGF), to a localized area of tissue using heparin sepharose-containing microcapsules in an amount effective to induce angiogenesis within the area of tissue. The angiogenic factors or expression plasmids encoding the factors are incorporated into the microcapsules as described in the working examples provided below for slow, sustained release into localized areas of tissue. In a particular embodiment, the microcapsules are made up of uncoated heparin sepharose beads, heparin sepharose beads coated with a single layer of alginate polymer, heparin sepharose beads coated with poly-ethylene glycol (PEG) polymer or heparin sepharose beads coated with alternating layers of alginate and PEG. Typically, the microcapsules range in size from 1-200 microns. As with angiogenic factors alone, suitable techniques for delivering the microcapsules include, for example, injection and surgical implantation. For example, injection can be performed using a catheter based trans-myocardial injection technology, such as the NOGA technology.

In still another embodiment, the present invention provides a method for promoting angiogenesis by contacting a localized area of tissue with a gradient of one or more angiogenic factors or a nucleic acid encoding one or more angiogenic factors, such that directed vascular growth along the gradient is achieved. Such directed vascular growth can be used to achieve interconnection and/or intraconnection of blood vessels (e.g., to circumvent blood flow around a blockage within a blood vessel).

In a particular embodiment, the angiogenic factor or nucleic acid is released in a gradient using a biocompatible material which is contacted with (e.g., implanted within) the localized area of tissue. The angiogenic factor is associated with the biocompatible material (e.g., absorbed onto the biocompatible material) such that it is released onto surrounding tissue. This can be achieved by treating the biocompatible material with the angiogenic factor prior to contact with (e.g., implantation into) a selected area of tissue. The angiogenic factor is then released from the biocompatible material onto the surrounding tissue in a directed gradient determined by the placement of the biocompatible polymer. Suitable biocompatible materials include, for example, polymers or threads which incorporate the angiogenic factor. In a preferred embodiment, the biocompatible material is an absorbable thread, such as polyglyconate monofilament, poliglecaprone 25-(Monocryl), polydiaxonone (PDS II), polyglactin 910, polyglycolic acid, Biodyn glycomer 631, chromic surgical gut or plain surgical gut.

Any suitable angiogenic factor can be delivered in accord with the methods of the present invention, including, for example, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, PDGF-BB, PDGF-AA, Angiopoietin 1, Angiopoietin 2, erythropoietin, BMP-2, BMP-4, BMP-7, TGF-beta, IGF-1, Osteopontin, Pleiotropin, Activin, Endothelin-1, and combinations thereof or an expression vectors encoding such angiogenic factors. Preferred angiogenic factors include PDGF-BB alone and in combination with another factor, such as an FGF factor (e.g., bFGF) or a VEGF factor. The angiogenic factors can be made or obtained from any source. For example, they can be purified from their native sources, or produced synthetically or by recombinant expression.

When administering angiogenic factors of the present invention in the form of an expression plasmid (e.g., by gene therapy), suitable vectors include, but are not limited to, adenoviral vectors, retroviral vectors, adeno-associated viral vectors, RNA vectors, liposomes, cationic lipids, lentiviral vectors and transposons. Lentiviral vectors which can be used to deliver the therapeutic factors of the invention include, for example, HIV, FIV, BIV, EIAV, and SIV.

These and other embodiments of the invention are described in the following figure detailed description, examples and figures.

### BRIEF DESCRIPTION OF THE FIGURES

FIG. 1 is a graph comparing levels of angiogenesis in the Matrigel model using a low dose of transduced cells encoding GFP alone (control), VEGF-A, VEGF-C, VEGF-D, bFGF or PDGF-BB. C57B1/10 mice were each injected subcutaneously into the abdominal with a low dose of 3×10⁵ retrovirally transduced autologous myoblast cells, suspended in 0.4 ml of Matrigel. Mice were sacrificed 13 days later and the Matrigel pellet and a section of the abdominal muscle adjacent to the pellet was removed. Samples were sectioned and the number of microvessels in the abdominal muscle was quantified by visual inspection of sections under the microscope. Shown is the number of microvessels per 10 high power fields counted. The most potent angiogenic effect was observed with VEGF-A, PDGF-BB and bFGF. Analysis of the dose response curve for PDGF-BB and VEGF-A transduced cells showed that PDGF-BB was more potent than VEGF-A at lower doses.

FIG. 2 is a graph comparing levels of angiogenesis in the Matrigel model using a high dose of cells transduced to express bFGF, VEGF-A and PDGF-BB. C57B1/10 mice were each injected with a high cell dose of 2×10⁶ retrovirally transduced autologous myoblast cells suspended in 0.4 ml of Matrigel. Mice were sacrificed 13 days later, the pellets were recovered, sectioned and the number of microvessels counted by visual inspection. Shown are the number of microvessels per 10 high power fields. At this cell dose, PDGF-BB was as potent as either bFGF or VEGF-A at stimulating angiogenesis.

FIG. 3 shows photographs of mouse corneas 6 days following the implantation of pellets coated with control saline (A), PDGF-BB (B), VEGF-A (C) or bFGF (D) alone. Bottom panels: Quantification of the angiogenic effect elicited by each factor. Vessel length (E), clock hours (F) and area (G) are shown.

FIG. 4(A) shows photographs of mouse corneas 6 days following the implantation of pellets coated with VEGF-A alone (left panel), bFGF (middle panel) or both factors combined (right panel). (B) shows the quantification of the angiogenic effect elicited by each growth factor in terms of clock hours (left panel), vessel length (middle panel) and area (right panel).

FIG. 5 (top panels) shows photographs of mouse corneas 6 days post-transplantation of pellets coated with bFGF alone (left panel) or bFGF combined with PDGF-BB (middle and right panels). Bottom panels show photographs of mouse corneas 6 days post-transplantation of pellets coated with either VEGF-A alone (left panel) or VEGF-A combined with PDGF-BB (right panel).

FIG. 6 is a graph comparing the quantification of angiogenesis in the mouse cornea model using PDGF-BB, VEGF-A or bFGF either alone or in combination. Corneal micropockets were created with a cataract knife in the eyes of 8-week old C57B1/6 mice. Into this pocket, aluminum sulfate pellets coated with between 80 and 160 ng of recombinant human PDGF-BB, VEGF-A, bFGF or combinations thereof were implanted and mice were monitored daily. A total of 5 mice were transplanted per group. The area of newly grown vessels was assessed 5 days post implantation. Mice implanted with control pellets showed no evidence of angiogenesis. When tested alone, bFGF stimulated the highest level of angiogenesis followed by VEGF-A and PDGF-BB. The level of angiogenesis stimulated by VEGF-A in combination with PDGF-BB was equivalent to that observed for bFGF alone. Unexpectedly, the most potent combination was PDGF-BB and bFGF. Of all combinations tested, PDGF-BB and bFGF together stimulated the greatest level of angiogenesis, significantly greater than that observed for VEGF-A and bFGF.

FIG. 7 is a schematic illustration of the experimental strategy to make heparin sepharose/alginate microcapsules. Heparin sepharose beads (Pharmacia: 50-150 μm in size) are mixed with a solution of sodium alginate to a final concentration of 200 mg/ml. The heparin sepharose/alginate solution is then loaded into a 5 ml syringe and slowly injected into a coaxial airflow system constructed at Genetix. The coaxial air flow creates a mist of the heparin sepharose/alginate solution which drops into a 1.5% calcium chloride bath. Once the alginate hits the calcium solution the alginate becomes cross-linked, forming a solid gel capsule roughly in the shape of a sphere. The size of the microcapsules can vary greatly from 50-400 μm. Large microcapsules (greater than 200 μm in size) are removed from the capsule mixture using a 200 μm sieve. Once formed the capsules are washed twice in sterile water and stored in buffer composed of 0.9% sodium chloride and 1 mM calcium chloride. Capsules are loaded with recombinant human PDGF-BB by incubation in binding buffer (0.9% NaCl, 1 mM CaCl2 and 0.05% gelatin) at 4° C. overnight (˜16 hours) with gentle shaking. The next day the capsules are removed, washed twice in binding buffer and either cultured in vitro to determine the kinetics of PDGF-BB release or injected in vivo to assess angiogenesis. The efficiency of PDGF-BB uptake is quantified by ELISA of the binding buffer following removal of the capsules.

FIG. 8 is a graph showing that heparin sepharose/alginate capsules bind large amounts of recombinant human PDGF-BB. Shown is the amount of PDGF-BB absorbed by 3000 capsules following incubation with various quantities of growth factor. The amount of PDGF-BB remaining in the binding buffer following incubation with capsules was quantified by ELISA. Three thousand capsules were able to absorb at least 35 μg of PDGF-BB representing 13 ng of PDGF-BB per capsule.

FIG. 9 is a graph showing that heparin sepharose/alginate microcapsules provide sustained and long term release of bound PDGF-BB at high levels in vitro. Ten μg of recombinant human PDGF-BB was incubated with three different types of test samples. The first test sample was composed of non-encapsulated heparin sepharose beads while the second and third groups were composed of alginate encapsulated heparin sepharose beads made using either a 1.2% or a 1.6% alginate solution.

FIG. 10 is a graph showing that PDGF-BB microcapsules potently stimulate angiogenesis in vivo in the stringent Matrigel model. Three thousand microcapsules loaded with 1 μg or 10 μg of PDGF-BB were mixed with 400 μl of Matrigel and subcutaneously injected into the abdominal region of C57B1/10 mice. Thirteen days later mice were sacrificed, the pellets and a section of the adjacent abdominal muscle was removed, fixed, sectioned and the number of microvessels quantified by visual inspection of the sections under the microscope.

FIG. 11 is a graph showing that PDGF-BB microcapsules stimulate angiogenesis in infarcted rat hearts 3 weeks post-injection. Infarcted rat hearts were injected with 1600 microcapsules containing μg (control) or 18 μg of PDGF-BB in a volume of 20 μl. Three weeks post injection rats were sacrificed, hearts were removed, fixed, sectioned and the number of microvessels within the infarct region quantified by visual inspection under a microscope. Shown is the number of microvessels per 5 high power fields for recipients of control and PDGF-BB microcapsules.

FIG. 12 shows an analysis of cardiac function in rats injected with control vs. PDGF-BB microcapsules following myocardial infarction. Left ventricular pressure (LVP), dP/dT, neg dP/dT and tau were measured prior to sacrifice at 3 weeks post injection. Left ventricular pressure (LVP) is the maximum pressure in the left ventricle during contraction. The dP/dT variable is the first derivative of the pressure wave and is separately viewed for the upstroke (dP/dT) and the downstroke (neg dP/dT). The upstroke (dP/dT) is a measure of contractility and reflects the condition of the muscle independent of the pressure. Neg dP/dT reflects the relaxation of the muscle, which together with the relaxation constant, tau, provides information on the stiffness of the ventricular wall following infarction. A significant improvement in all parameters was detected in rats injected with PDGF-BB microcapsules.

FIG. 13 is a graph showing that PDGF-BB and bFGF delivered by slow release microcapsules potently synergize to stimulate angiogenesis in vivo in the stringent Matrigel model. Three thousand microcapsules loaded with 1 μg of bFGF were mixed with 400 μl of Matrigel and subcutaneously injected into the abdominal region of C57B1/10 mice. Thirteen days later mice were sacrificed, the pellets and a section of the adjacent abdominal muscle was removed, fixed, sectioned and the number of microvessels quantified by visual inspection of the sections under the microscope.

FIG. 14 is a schematic illustration of the structure of various angiogenic expression plasmids. All vectors were constructed using the pCI vector backbone from Promega. All vectors contained the Cytomegalovirus immediate-early enhancer/promoter region, a chimeric intron and the late poly adenylation signal from SV40. The cDNA encoding either human PDGF-BB, VEGF-A or bFGF was inserted into this vector downstream of the chimeric intron. A cDNA encoding for the mature PDGF-BB protein was cis-linked to the secretory signal from the murine IgG kappa immunoglobulin light chain gene while the VEGF-A cDNA utilized its endogenous secretory signal. The bFGF cDNA was linked in cis to the secretory signal from the human Interleukin-2 cDNA. The level of angiogenic protein secreted from transiently transfected 293T cells, as assessed by ELISA, is shown to the right.

FIG. 15 is a comparison of angiogenic features of the PDGF family Micropellets of PDGF-AA (a), PDGF-BB (b) or PDGF-AB (c) were implanted into corneal micropockets of C57BL/6 mice. Corneal neovascularization was measured on day 5 after growth factor implantation. Arrows point to the implanted pellets. Photographs represent 20×amplification of the mouse eye. Quantification of corneal neovascularization is presented as maximal areas of neovascularization (e). Graphs represent mean values (±SEM) of 11-16 eyes (6-8 mice) in each group. Nylon meshes containing PDGF-BB (g) or BSA (t) were implanted on CAMs of 6-d-old chick embryos. After 6-day implantation, the formation of new blood vessels was examined under a stereoscope. A CAM with a methylcellulose mesh containing BSA alone served as a negative control (f). New blood vessels and sprouts are marked with arrows in g. M=mesh.

FIG. 16 shows synergistic angiogenesis induced by bFGF and PDGF-BB. Micropellets containing no growth factor (a), 160 ng PDGF-BB (b), 160 ng VEGF (c), 160 ng PDGF-BB plus 160 ng VEGF (d), 80 ng FGF-2 (e), or 160 ng PDGF-BB plus 80 ng FGF-2 (f) were implanted into corneal micropockets of C57B16/J mice. Corneal neovascularization was examined on day 5 after growth factor implantation. Arrows point to the implanted pellets. Photographs represent 20×amplification of the mouse eye. Quantification of corneal neovascularization is presented as maximal vessel areas of neovascularization (g and h). Graphs represent mean values (±SEM) of 12-16 eyes (6-8 mice) in each group. Slow release microcapsules containing PDGF-BB alone, FGF-2 alone, or PDGF-BB plus FGF-2 was subcutaneously injected into the abdominal region of C57BL/6 mice. Neovascularization was quantified by counting microvessels in histological sections under a microscope (i). At least 10 different fields were randomly counted.

FIG. 17 shows stability of blood vessels induced by micropellets containing 160 ng PDGF-BB, 40 ng FGF-2, 160 ng PDGF-BB plus 40 ng FGF-2, 160 ng VEGF, or 160 ng PDGF-BB plus 160 ng VEGF. Micropellets were implanted into mouse corneal micropockets. The corneal neovascularization was examined and photographed at the indicated time points. Arrows in indicate the implanted pellet. Asterisks indicate positions of pellets in those corneas that lost implanted pellets.

FIG. 18 shows corneal neovascularization after depletion of angiogenic factors. Angiogenic factors were implanted into corneal micropockets of C57BL/6 mice. Ten to twelve corneas were used in each group. At day 6 after implantation, the implanted angiogenic factors were removed. The corneal neovascularization was examined and photographed at the indicated time points. Arrows indicate the implanted pellet. Asterisks indicate former positions of removed pellets.

FIG. 19 shows graphs of vessel Maturation Index as percentages of mural positive vessels at day 5 (a), day 12 (b), and day 25 (c). Results are presented as mean determinants (±SEM) of 6-8 serial sections in each group (20×).

FIG. 20 shows stimulation of collaterogenesis and improvement of blood perfusion by dual delivery of FGF-2/PDGF-BB. Panels a-d show day 23 after ligation of femoral artery (position marked with asterisks), angiograph analysis of ischemic hind limbs of PBS buffer- (a), FGF-2- (b), PDGF-BB- (c) and FGF-2/PDGF-BB- (d) treated groups. Arrows in panels b-d point to newly formed collaterals and arrowheads in b and d point to a direct comparison of vessel dilation of FGF-2- and FGF-2/PDGF-BB-induced collaterals. Panels f-n show anti-α-SMA staining of histological sections of PBS buffer- (f and g), FGF-2- (h and i), PDGF-BB- (and k) and FGF-2/PDGF-BB- (1-n) treated ischemic hind limb muscle tissues. Arrows in f-n point to newly formed arterial vessels. Panel o shows quantification of large vessel lumen areas (>700 μm²) as % of total vessel lumen areas. Panel e shows laser Doppler analysis of hind limb paw blood perfusion.

FIG. 21 shows in situ detection of PDGFR-α and -β on newly formed blood vessels. Mouse corneas implanted with FGF-2 (a,c and e) or PDGF-BB (b and d) were removed at day 5 after implantation. Bright-field photomicrographs of emulsion autoradiograms of corneal tissue sections hybridized with the oligonucleotide probes for mouse PDGFR-α (a and b) and -β (c and d) show labeled vascular endothelial cells and smooth muscle cells. A 50-mer random probe was used as a negative control in detection of FGF-2-induced corneal vessels (e). Panel f shows a schematic representation of the role of FGF-2/PDGF in blood vessel stability.

FIG. 22 shows the effect of PDGF-BB on heart tissue remodeling by impovement in endocardial regional wall motion with no increase in normalized wall thickening. Panel (a) shows the change in the extent of target area with reduced endocardial motion at stress. Panel (b) shows a similar result when the ratio of AUCtarget/AUCnon-target was used as measure of regional wall motion.

### DETAILED DESCRIPTION OF THE INVENTION

While growth promoting factors have been described as angiogenic agents, the efficacy of such proteins as therapeutic agents for the treatment of tissue ischemias, such as peripheral, pulmonary, and/or myocardial ischemia, has not yet been demonstrated.

In particular, promotion of vascular networks that remain stable for a sustained period following administration of such growth factors has not yet been achieved. The present invention provides, for the first time, proven methods for promoting angiogenesis and/or tissue remodeling to treat tissue ischemias using particular angiogenic factors, such as PDGF-BB, as well as particular combinations of factors (e.g., combinations which include PDGF-BB, such as the combination of PDGF-BB and bFGF). The present invention also provides improved methods for delivering such factors to increase their therapeutic efficacy, for example, by enabling directed and/or controlled release of the angiogenic factors onto surrounding tissue (e.g., ischemic myocardium).

As used herein, the term “ischemia” refers to any localized tissue anemia due to obstruction of the inflow of blood. Tissue ischemia can include, but is not limited to, pulmonary ischemia, limb ischemia, brain ischemia, retinal ischemia, nerve tissue ischemia, kidney ischemia, skin ischemia, subcutaneous tissue ischemia, ischemia of the gut and ischemia of the brain. “Peripheral ischemia,” as used herein, refers to ischemia found in areas distal to or away from the center of the body, such as tissue of the limbs. “Myocardial ischemia” refers to circulatory disturbances caused by coronary atherosclerosis and/or inadequate oxygen supply to the myocardium. “Brain ischemia” or “ischemia of the brain” refers to a disturbance of the blood supply to an area of the brain. “Retinal ischemia” refers to ischemia found in the retina of the eye. “Nerve tissue ischemia,” as used herein, refers to circulatory disturbances found in nerve tissue. “Kidney ischemia” refers to decreased oxygenation and disturbance of blood supply to renal tissues. “Skin ischemia” refers to decreased oxygenation and disturbance of blood supply to the skin. “Subcutaneous tissue ischemia” refers to decreased oxygenation and disturbance of blood supply below the skin. “Ischemia of the gut” refers to ischemia found within the digestive system, including death of part of the intestine following cessation in its blood supply, which often results from narrowing of the supplying artery.

As used herein, the term “angiogenesis” refers to the promotion of growth of new blood vessels from existing vasculature. The term “tissue remodeling” refers to the reformation of existing vasculature. The term “localized” refers to a selected area of tissue to which angiogenic factors and and/or genes encoding the factors are delivered.

Angiogenic Factors

As used herein, the term “angiogenic factor” or “angiogenic protein” refers to any known protein (“factor”) capable of promoting growth of new blood vessels from existing vasculature (“angiogenesis”). Suitable angiogenic factors for use in the invention include, for example, PDGF-BB, PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof. The term PBGF-B as used herein encompasses both PDGF-B and PDGF-BB.

Angiogenic factors can act independently, or in combination with one another. When in combination, angiogenic factors can also act synergistically, whereby the combined effect of the factors is greater than the sum of the effects of the individual factors taken separately. For example, as part of the present invention it was discovered that the angiogenic factors PDGF-B and bFGF act synergistically to promote angiogenesis and/or cause tissue remodeling.

The term “angiogenic factor” or “angiogenic protein” also encompasses to functional analogues of such factors. Functional analogues include, for example, functional portions of the factors. Functional analogues also include anti-idiotypic antibodies which bind to the receptors of the factors and, thus, mimic the activity of the factors in promoting angiogenesis and/or tissue remodeling. Methods for generating such anti-idiotypic antibodies are well known in the art and are described, for example, in WO 97/23510, the contents of which are incorporated by reference herein.

Antigens have specific epitopes to which certain antibodies will bind. The region of the antibody that specifically interacts with the epitopes on the antigen is called the antigen combining site. The antigen combining site is composed of a collection of idiotopes which are unique sequences on the antibody that specifically interact with the epitopes on the antigen. The specific collection of idiotopes that interact with the epitopes on the antigen is defined as an antibody's “idiotype”. Accordingly, an anti-idiotype antibody is an antibody that is directed against the antigen combining site of the first antibody. Anti-idiotype antibodies combine with those specific sequences and may resemble or act as the epitope to which the first antibody reacts. For example, one can have an antibody that binds to specific epitopes on bFGF. One can then make a second antibody (an anti-idiotype antibody) that specifically interacts with antigen combining site of the first antibody. This anti-idiotype antibody may then mimic the biological activity of bFGF itself by binding to the bFGF receptor and activating it.

The use of anti-idiotype antibodies (as growth factor analogues) in the methods of the present invention can be advantageous in that many growth factors have a very short half life and therefore most of the factor that is given to a patient is not utilized. In contrast, antibodies have much greater half lives and therefore their potency is maintained for a greater length of time in vivo. In addition the levels of growth factors that are required to achieve a biological effect can, in certain instances, produce adverse reactions such as toxicity and hypotension. Since lower levels of anti-idiotype antibodies may be required to produce the same biological effect these adverse side effects may be prevented.

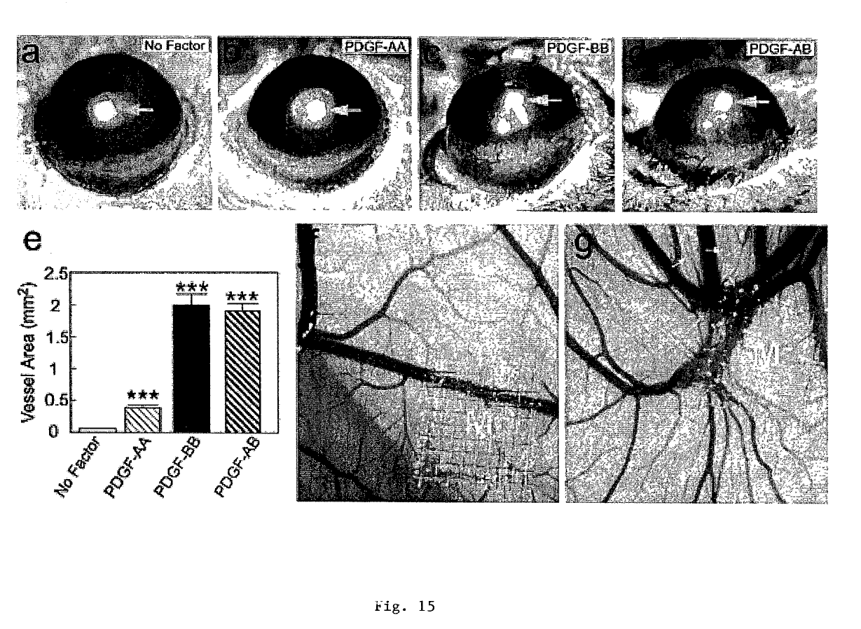

Angiogenic factors used in the present invention can be produced or obtained from any suitable source. For example, the factors can be purified from their native sources, or produced synthetically or by recombinant expression. The factors can be administered to patients as a protein composition. Alternatively, the factors can be administered in the form of an expression plasmid encoding the factors, as is described in further detail below. The construction of suitable expression plasmids is well known in the art. Particular angiogenic expression plasmids for use in the invention are shown in FIG. 14 and are described in Example 1. Suitable vectors for constructing expression plasmids include, for example, adenoviral vectors, retroviral vectors, adeno-associated viral vectors, RNA vectors, liposomes, cationic lipids, lentiviral vectors and transposons.

Accordingly, in a further embodiment of the invention, nucleic acids which encode angiogenic factors of the present invention can be inserted into vectors and used as gene therapy vectors. In one embodiment, the gene therapy vector is a viral vector, e.g. replication defective retroviruses, adenoviruses and adeno-associated viruses, wherein the nucleic acid molecule encoding the photosensitive protein is ligated into the viral genome. Viral vectors, including lentiviral, retroviral, and adeno-associated virus vectors, are generally understood to be the gene therapy vector of choice for the transfer of exogenous genes in vivo, particularly into humans. Examples of lentiviral vectors include, but are not limited to, HIV, FIV, BIV, EIAV, and SIV. Viral vectors provide efficient delivery of genes into cells, and the transferred nucleic acids are stably integrated into the chromosomal DNA of the host.

The infectivity of the viral vector can be made cell-specific by expressing cell-specific proteins on the surface of the viral particle which will interact with receptors unique to the cell of interest. In this manner, the viral vector can be targeted to retinal ganglion cells. Expression is further enhanced by the use of tissue-or cell-specific transcriptional regulatory sequences which control expression of the gene.

The step of facilitating the production of infectious viral particles in the cells may be carried out using conventional techniques, such as standard cell culture growth techniques. The step of collecting the infectious virus particles also can be carried out using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art. Alternatively, the viral vectors of the invention can be administered ex vivo or in vitro to cells or tissues using standard transfection techniques well known in the art.

Other methods relating to the use of viral vectors in gene therapy can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Accordingly, in one embodiment, the present invention provides novel methods and compositions for promoting angiogenesis to treat a variety of tissue ischemias. Selected angiogenic factors or synergistic combinations of factors, functional analogues of such factors or combinations of factors, or nucleic acids encoding such factors or combinations of factors, are delivered to a localized area of tissue in an amount effective to induce angiogenesis within the area of tissue.

In a particular embodiment, the angiogenic factor or combination of factors comprises PDGF-BB. A preferred combination of factors comprises PDGF-BB and bFGF. As shown herein, PDGF-BB and bFGF exhibit an unexpected synergistic effect in promoting angiogenesis, e.g., in ischemic myocardium, when delivered using a suitable delivery system, such as NOGA injection device.

Delivery of Angiogenic Factors

As discussed above, the present invention is based on the finding that delivery of particular angiogenic factors and combinations of factors to ischemic tissue promotes angiogenesis and/or beneficial tissue remodeling within the affected area. Accordingly, the invention can be achieved by delivering such factors using any suitable technique for contacting proteins in vivo with localized areas of tissue.

In one embodiment, the angiogenic factors are delivered directly to a selected area of tissue. As used herein, the term “direct” refers to direct placement of the factors onto or into the affected tissue, as opposed to, for example, indirect delivery through the bloodstream. By way of illustration, when delivering factors to areas of ischemic myocardium, the factors can be delivered though an adjacent body lumen (e.g., vessel) wall and directly injected into the myocardial tissue on the other side. This can be achieved, for example, using a NOGA delivery device or similar system.

Angiogenic factors also can be delivered alone or in association with a material or composition which provides for controlled, sustained release of the factors onto surrounding tissue. As used herein, the terms “controlled-release”, “sustained release” and “slow release” are used interchangeably, and refer to the medication of the rate at which the factors are delivered such that they are released for a prolonged period of time. One problem in using purified (e.g., recombinant) angiogenic proteins to stimulate angiogenesis for the treatment of myocardial and peripheral tissue ischemia can be the short half-life of the protein upon injection in vivo. Accordingly, in further embodiments, the invention provides a variety of improved techniques for delivering angiogenic factors, including genes encoding the factors, to selected areas of tissue in controlled, sustained fashion.

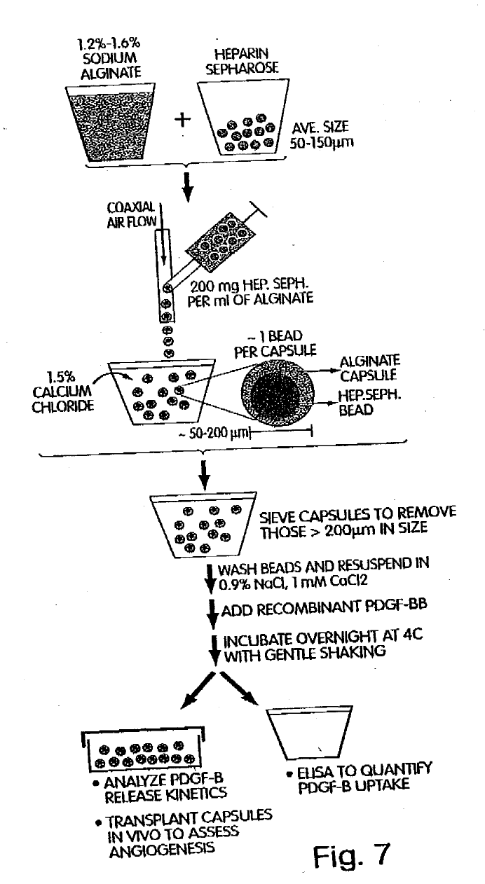

In one embodiment, this is achieved using slow-release heparin sepharose-containing microcapsules. One property that many angiogenic factors share is the ability to bind to heparin, a highly sulfated glycosaminoglycan that plays a role in anti-coagulation in vivo. This property is exploited in making the heparin sepharose containing microcapsules of the present invention which bind angiogenic factors and provide slow release of the factors when contacted (e.g., implanted) with localized tissue. This is described in detail and illustrated, for example, in Example 3 below, and shown schematically in FIG. 7.

The heparin sepharose-containing microcapsules of the invention can be delivered to affected tissue by, for example, injection or surgical implantion. In a particular embodiment, the microcapsules are delivered to a localized area of tissue using the NOGA system (Biosense). NOGA is a 3 dimensional catheter based transmyocardial injection system. A catheter is inserted into a major vein/artery and is snaked up into either of the ventricles of the heart. The end of the catheter contains a needle and a space in which therapeutic agents can be inserted and subsequently injected intra-myocardially into the damaged areas of the heart muscle. The NOGA delivery method is much easier and safer for the patient and is much more efficacious than other methods of delivery including intracoronary injections or placing therapeutic agents against the wall of the heart. Due to the nature of the system, only therapeutic agents which can be physically delivered through a 25-27 gauge needle can be used. Thus macrocapsules described in the prior art cannot be used with such a system, whereas microcapsules of the present invention can be used with the system.

The microcapsules are composed of single heparin sepharose beads which optionally can be coated with a thin layer of alginate polymer. In a particular embodiment of the invention, the microcapsules are made up of uncoated heparin sepharose beads, heparin sepharose beads coated with a single layer of alginate polymer, heparin sepharose beads coated with poly-ethylene glycol (PEG) polymer or heparin sepharose beads coated with alternating layers of alginate and PEG.

Using, for example, the coaxial air flow technology shown in FIG. 7 and described in Example 1, the microcapsules can also be made small enough for use in the NOGA delivery system. For example, the microcapsules typically range from 1-200 microns in size. Moreover, they are able to absorb large quantities of angiogenic factors, such as FGF-2, VEGF-A and PDGF-BB, and slowly release the factors over extended periods of time at levels which are able to stimulate the growth of new blood vessels in vivo.

In another embodiment, controlled, sustained release of angiogenic factors and/or genes is achieved by applying a gradient of the factors or genes to selected tissue, such that directed vascular growth along the gradient is achieved. This stimulates chemotaxis and proliferation of endothelial cells and their supporting cells along the gradient, towards the source of the factor. Regulating the growth of new vessels from existing vasculature (angiogenesis) that effectively bypass an arterial lesion requires strict spatial and temporal control. Accordingly, by forming a directed gradient of angiogenic factors in accordance with the present invention, interconnection and/or intraconnection of blood vessels (e.g., to circumvent blood flow around a blockage within a blood vessel) can be achieved.

In a particular embodiment, the angiogenic factor or genes is released in a gradient using a biocompatible material which contains the factor such that the factor is released onto surrounding tissue when the biocompatible material is contacted with (e.g., implanted within) the tissue. This can be achieved by treating the biocompatible material with the angiogenic factor prior to contact with (e.g., implantation into) a selected area of tissue in a manner which allows for release of the factor from the material in vivo. The biocompatible material is then contacted with (e.g., implanted into) a localized area of tissue in a configuration which provides a directed gradient of the angiogenic factor once it is released from the material.

Suitable biocompatible materials for use in the invention include, for example, polymers or threads which incorporate the angiogenic factor. In a preferred embodiment, the biocompatible material is an absorbable thread, such as polyglyconate monofilament, poliglecaprone 25-(Monocryl), polydiaxonone (PDS II), polyglactin 910, polyglycolic acid, Biodyn glycomer 631, chromic surgical gut or plain surgical gut. The biocompatible material can be coated with one or several angiogenic factors to allow delivery of growth factor or growth factor combinations that provide the optimal angiogenic stimulus. The biocompatible material can also be engineered to release certain growth factors at specific rates and at specific times that may help mimic the natural angiogenic process more closely.

The invention is further illustrated by the following examples which should not be construed as limiting.

### EXAMPLES

Materials and Methods

Reagents, Cells, and Animals

Recombinant PDGF-AA, PDGF-BB and PDGF-AB were obtained from R&D Systems Inc. (Minnesota, Minn.). Recombinant human VEGF₁₆₅ was prepared as previously described Schollmann et al. (1992) J. Biol. Chem. 267:18032-18039. Recombinant human FGF-2 was obtained from Pharmacia & UpJohn (Milan, Italy). Male 5-6-wk-old C57BL/6 mice were acclimated and caged in groups of six or less. Animals were anaesthetized by injection of a mixture of dormicum and hypnorm (1:1) before all procedures and killed with a lethal dose of CO₂.

Chick Chorioallantoic Membrane (CAM) Assay

The CAM assay is one of the most widely used in vivo angiogenesis assays which detects angiogenic activity of compounds during embryonic development (Jain et al. (1997) Nat. Med. 3:1203-1208). Three-day-old fertilized white Leghorn eggs (OVA Production, Sörgården, Sweden) incubated at 37° C. were cracked and chick embryos with intact yolks were carefully placed in 20×100 mm plastic petri-dishes. After 6 days of incubation in 4% CO₂ at 37° C., discs of methylcellulose containing 2.5 μg of PDGF-BB or bovine serum albumin (BSA) dried on a nylon mesh (3×3 mm) were implanted on the CAM of individual embryos. The nylon mesh discs were made by desiccation of 10 μl of 0.45% methylcellulose in H₂O. After 4-6 days of incubation, embryos and CAMs were examined for the formation of new blood vessels around the field of the implanted discs by a stereoscope. Discs of methylcellulose containing BSA were used as negative controls. In general, CAM assay experiments were carried out three times using II embryos for each experiment.

Mouse Corneal Micropocket Assay

The mouse corneal assay was performed according to procedures previously described in Cao et al. (1990) Proc. Natl. Acad. Sci. USA 95: 14389-94. Corneal micropockets were created with a modified von Graefe cataract knife in both eyes of each male 5-6-wk-old C57BL/6 mouse. A micropellet (0.35×0.35 mm) of sucrose aluminum sulfate (Bukh Meditec, Copenhagen, Denmark) coated with hydron polymer type NCC (IFN Sciences, New Brunswick, N.J.) containing the molecule of interest, e.g. VEGF, PDGF-BB, -AA or -AB, was surgically implanted. Half amounts of FGF-2 were used in combinatorial experiments. The pellet was positioned 1.2-1.5 mm from the corneal limbus. After implantation, erythromycin/ophthalmic ointment was applied to each eye. Eyes were examined by a slit-lamp biomicroscope on indicated days after pellet implantation. Vessel lengths and clock hours of circumferential neovascularization were measured under a stereoscope. In some experiments, the implanted pellets were removed on day 6 after implantation.

Preparation of Heparin Sepharose Beads

Heparin-sepharose beads (Pharmacia, Piscataway, N.J.) were sterilized for 30 min by exposure to UV irradiation. Heparin sepharose beads were mixed with 1 μg of human PDGF-BB and/or 1 μg FGF-2 (Peprotech, Rocky Hill, N.J.) and were incubated overnight with gentle rocking at +4° C. in binding buffer composed of 0.9% sodium chloride, 1 mM calcium chloride, 0.05% gelatin. The following day, the binding buffer was removed and analyzed by ELISA (R & D Systems, Minneapolis, Minn.) to quantify the amount of PDGF-BB adsorbed by the beads.

Matrigel Assay

Heparin-sepharose beads, with or without growth factors, were washed twice in a binding buffer, mixed with Matrigel and injected subcutaneously into the abdominal region of anesthetized male 5-6-wk-old C57BL/6 mice. Three thousand beads, suspended in 400 μl of Matrigel (Becton Dickinson, San Diego, Calif.), were injected per mouse. Two weeks post-injection mice were sacrificed, the Matrigel pellets were retrieved, fixed, sectioned and analyzed for evidence of angiogenesis by visual inspection of pellet sections under the microscope.

Rat Hind Limb Ischemic Model

The rat ischemic hind limb model was carried out according to a two-stage procedure as previously described in Seifert et al. (1985) J. Cardiovasc. Surg. (Torino) 26:502-508. This procedure created ischemia in the left hind limb with the right leg as a control. The first operation was performed through a midline lapratomy. Under a dissecting microscope, all left side branches of aorta distal to the renal arteries and all left side branches of iliac artery were ligated with 6-0 resorbable suture. These ligated vessels include the spermatic, left lumbar, ileolumbar, inferior mesenteric, caudal arteries and all branches from the left iliac artery down to the inguinal ligament. Five days later, the rats were subjected to a second operation. The femoral artery was ligated, by a left inguinal incision at a position close to the origin of the superficial epigastric artery, which was subsequently ligated. A slightly less severe ischemic condition was created as compared to that previously described by cutting the superficial epigastric artery and preserving the superficial circumflex artery. Animals were randomly divided into 4 groups (5 rats/each) and treated with FGF-2, PDGF-BB, FGF-2/PDGF-BB and PBS. On the same day of the second operation, growth factors in the same slow release polymers as described in the mouse corneal model. (800 ng FGF-2, 1600 ng PFGF-BB or 800 ng FGF-2/1600 ng PDGF-BB) were implanted into intramuscular pockets near the ligation sites. After completion of the operation, soluble growth factors (1.5 μg FGF-2, 3 μg PDGF-BB or 1.5 μg FGF-2/3 μg PDGF-BB) in 400 μl PBS were injected into 3 sites close to the ligation sites and continued every other day for totally 12 days.

Laser Doppler Imaging

A moorLDI-VR (Visible red laser Doppler imager, Moor instruments Ltd, Axminister, England) was used to assess limb blood perfusion (Essex et al. (1991) J. Biomed. Eng. 13:189-194). At day 23 after the second operation, blood flow in the ischemic hind paws and in the healthy control paw of each animal were examined. Mean flux values were calculated using the MooreLDI Image processing V3.04 Software. Improvements of blood flow were calculated as the average of percentages of blood flow in ischemic paw as compared with the healthy paw and subtracted from the values of the PBS-treated group.

Angiography

Rats were anesthetized and the proximal abdominal aorta was ligated trough a midline incision. The aorta was cannulated (PE50) distal to the ligature and the tip of catheter was inserted into the left iliac artery. Rats were positioned supine on an image plate (Agfa), directly on the collimator of a mobile x-ray system. First 0.7 ml of papaverin (4 mg/ml) was injected followed by 0.5-0.8 ml of Visipaque®. An image was obtained immediately after contrast injection.

Immunohistochemistry

The growth factor-implanted mouse eyes were enucleated at day 5 after implantation and immediately frozen on dry ice and stored at −80° C. before use. Frozen sections of 10 μm were cut using a cryomicrotome. Sections were air-dried for 10 min, fixed with acetone and blocked with 30% non-immune goat serum. Endogenous biotin was blocked by using an avidin-biotin reagent (Vector laboratories, Burlingame, USA). A mixture of primary antibodies consisting of rat anti-mouse CD31 (1:100, Pharmingen, San Diego, USA) and mouse anti-human desmin (1:50 NOVO Castra, Newcastle upon Tyne, UK) were added and incubated for 1 h at room temperature. After repeated washing, secondary antibodies of rabbit anti-rat-FITC (Dako A/S,Glostrup, Denmark) and biotinylated goat anti-mouse IgG (Southern Biotechnology Associates Inc., Birmingham, USA) were added to the tissue sections before incubation for 30 min. Following rigorous rinsing, streptavidin-conjugated Cy3 (1:2500, Jackson ImmunoResearch, West Grove, USA) was added to samples and incubated for 30 min. After washing in phosphate buffered saline, slides were mounted in 90% glycerol and examined under a fluorescent microscope (Nikon) at 20×magnification. Images were collected with a digital camera system, and further analyzed with Adobe Photoshop 6.0 software program.

In the rat ischemic model, muscle tissues from the ischemic and healthy hind limbs were dissected and fixed in 3% PFA, embedded in paraffin, and cut at the thickness of 5 μm cross sections. After deparaffinization, a mouse anti-smooth muscle actin (1:400, Sigma) was incubated overnight at 4° C. A secondary anti-mouse antibody labeled with horse radish peroxidase 1:200 (HRP) was incubated for 30 min at RT. The reaction was developed by addition of DAB substrate. Microphotographs were taken under a microscope (4×). Lumen areas were calculated on the micrographs (>6/group) using a stereological approach with a square grid (d=2 mm). Total lumen areas were calculated as the sum of all measurable vessel lumens in a field, and large vessels were defined with a cutoff>700 μm².

In situ Hybridization

In situ hybridization was carried out according to a standard method using radiolabeled oligonucleotide probes and high stringency conditions. Two probes complementary to PDGFR-α (nucleotides 423-470 and 3083-3130) and two probes complementary to PDGFR-β (946-996 and 2610-2657) were used (Carmeliet et al. (2001) Nat. Biotechnol. 19:1019-1020; Smits et al. (1989) Growth Factors 2:1-8). All probes were used separately and did not match any known sequence in Genbank except those of the intended genes. A control 50-mer random probe not complementary to any sequence deposited in Genbank, was also used. Following 3′ end-labeling with [³³P]dATP (NEN Dupont) by terminal deoxynucleotidyl transferase (Amersham), probes were purified (QIAquick™ Nucleotide Removal Kit Protocol, QIAGEN). Slides were incubated overnight (42° C.) with 0.1 ml hybridization cocktail, containing 50% formamide, 4×SSC (0.15M NaCl, 15 mM sodium citrate pH 7.0), 1×Denhardts solution, 1% Sarcosyl, 0.02M Na₃PO₄ pH 7.0, 10% dextransulphate, 0.06M DTT, 0.1 mg/ml sheared salmon sperm DNA and hot probe. Slides were then rinsed four times (45 min) in 1×SSC at 60° C. and allowed to adjust to room temperature during a fifth rinse in 1×SSC. Further rinsing was carried out in distilled water and increasing concentrations of ethanol. Air-dried slides were then dipped in emulsion (Kodak NTB2, diluted 1:1 with water). After five weeks of exposure, slides were developed, counterstained with cresyl violet and mounted (Entellan, Merck). The control probe was hybridized and processed together with the other probes and gave rise to no specific pattern of hybridization signals in the mouse tissue. Specific labeling was confirmed by similar expression patterns revealed by two probes (complementary to different parts of the mRNA) for each PDGFR-α and PDGFR-β. Detection of positive autoradiographic signals was based on serial observations of adjacent sections from each tissue specimen, and accumulation of silver grains in the emulsion above specific cells and tissues identified by the staining procedures. Only cells over which silver grain accumulation was clearly above surrounding background levels and could be confirmed by both dark-field and high magnification bright-field, were regarded as positive.

Statistics

Statistical evaluation of the results was made by two-tailed Student t-test with INSTAT 1.1 and Microsoft EXCEL 5 programs, and by one-way analysis of variance (ANOVA) followed by post hoc test (Newman-Keuls). To relate the effects of individual and combined treatments, the effects of PDGF-BB and FGF-2 applied alone were summarized and compared with the effect of PFGF-BB and FGF-2 applied together using Mann-Whitney U-test. The same method was used to compare the effects of PDGF-BB and VEGF used alone or together.

### Example 1

### Construction of Retroviral Vectors Containing Angiogenic cDNAs Analysis of Virus Titer and Assessment of Stable Gene Transfer

In order to provide stable, high-level delivery of PDGF-BB in the Matrigel model, primary myoblasts from C57B1/10 mice were transduced with retroviral vectors encoding human PDGF-BB. To compare the angiogenic potential of PDGF-BB to other known angiogenic agents, retroviral vectors encoding human VEGF-A165, VEGF-C, VEGF-A, VEGF-D, PDGF-BB, or bFGF also were constructed and tested. All vectors are shown schematically in FIG. 14. Since VEGF-C, VEGF-D and PDGF-BB cDNAs encode proteins which are inactive (or less active in the case of PDGF-BB) in their non-processed form, vectors containing cDNAs encoding the mature forms of the aforementioned proteins linked to the powerful secretory signal from the murine IgG kappa immunoglobulin gene were constructed.

All vectors also contained the gene encoding the green fluorescent protein (GFP) to enable the fast efficient and non-toxic selection of transduced target cells by fluorescence activated cell sorting (FACS). FACS sorting is used to isolate the brightest 10% of GFP positive retrovirally transduced cells. Since both GFP and the angiogenic CDNA are translated from the same mRNA molecule, this ensures that the sorted cells also express high levels of the angiogenic protein. A strong correlation between the fraction of top GFP expressing cells and the amount of angiogenic protein secreted by the sorted cells exists. This result, in combination with data from Southern blot analysis of transduced cells which showed 3-6 proviral copies per genome in sorted GFP positive cells, indicates that the levels of angiogenic protein production and secretion are at their maximal level using this system.

All vectors were tested for stability of gene transfer and virus titer. Vectors demonstrated a virus titer ranging from approximately 5×10⁵-1.2×10⁶ infectious virus particles per ml and all vectors showed stable transfer of the angiogenic cDNA to target primary skeletal muscle myoblasts from C57B1/10 mice. To enable the easy localization and quantification of transduced myoblasts following injection in vivo, all myoblast samples were also marked by infection with a retroviral vector encoding a β-galactosidase/neomycin (β-GEO) resistance fusion gene.

Characterization of the Protein Expression and Secretion Properties of Retrovirally Transduced C57B1/10 Myoblasts

High-level expression and secretion of the encoded angiogenic proteins from transduced myoblast cells was demonstrated by either Western blot or ELISA analysis of supernatants from virally transduced myoblasts.

| | Amount of Protein Secreted |
|---|---|
| Angiogenic Factor | (ng/1 × 10⁶ cells/24 hours) |
| VEGF-A | 88 |
| VEGF-C | 790 |
| VEGF-D | 580 |
| PDGF-BB | 56 |
| bFGF | 20 |

### Example 2

### Comparison of Angiogenic Stimulation Among Various Factors by Matrigel Assay

The potency of the angiogenic proteins secreted from the transduced myoblasts described in Example 1 was assessed in vivo using the stringent Matrigel assay. In brief, 3×10⁵-2×10⁶ transduced myoblasts were suspended in Matrigel and injected subcutaneously into the dorsal abdominal region of C57B1/10 mice. The Matrigel pellets, in addition to a section of abdominal muscle adjacent to the Matrigel pellet, were recovered 13 days post-injection. Following harvesting of the Matrigel pellet and the adjacent abdominal muscle 13 days post-injection, Matrigel pellets were stained with X-gal and analyzed for the presence of blue, retrovirally transduced myoblasts. In addition, the number of microvessels in the adjacent abdominal muscle was quantified by visual inspection.

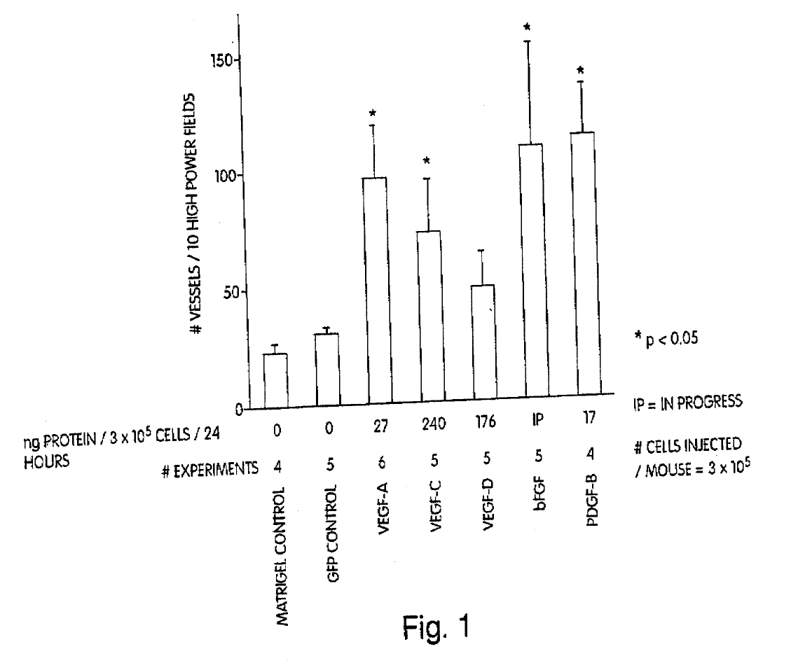

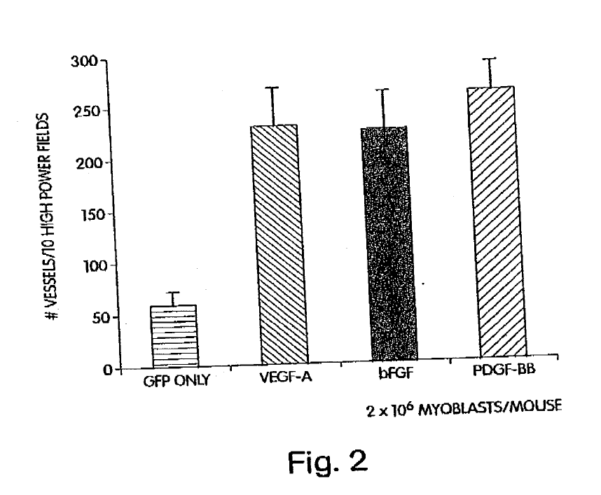

A significant (p<0.05) angiogenic response was observed for VEGF-A, VEGF-C, PDGF-BB, and bFGF (see FIGS. 1 and 2). Transplantation transduced myoblasts secreting the aforementioned growth factors resulted in approximately a 4-5-fold increase in the number of microvessels observed. The most potent angiogenic response was observed for PDGF-BB, followed by VEGF-A and bFGF (FIGS. 1 and 2).

### Example 3

### In vivo Assessment of Angiogenesis

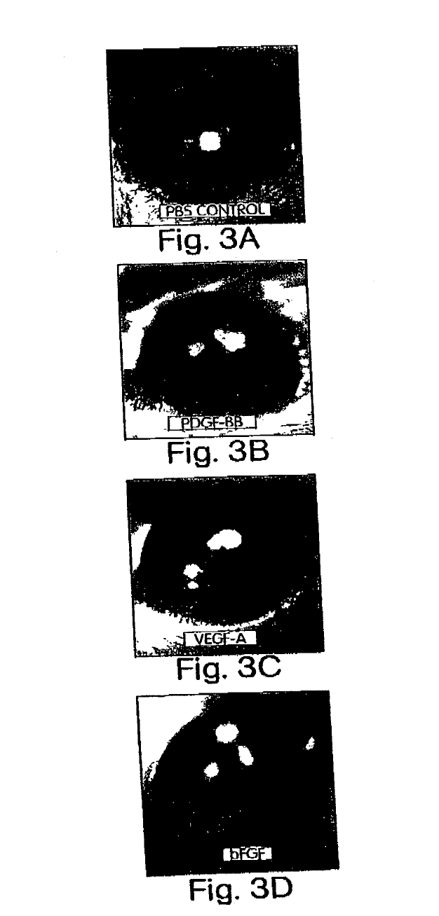

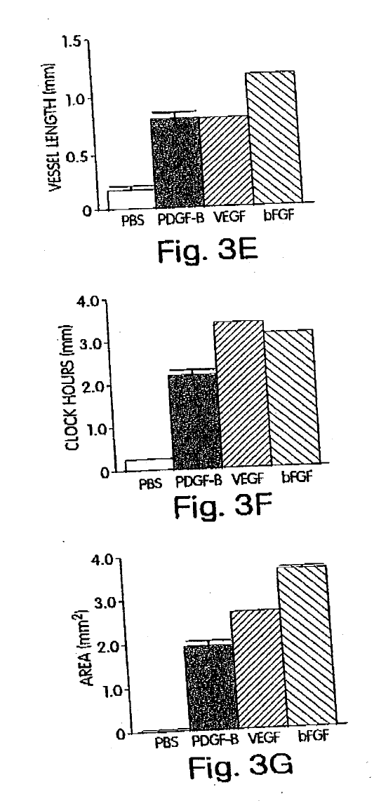

The angiogenic potential of selected growth factors was investigated using the mouse cornea model. Corneal micropockets were created with a cataract knife in the eye of 8-week old C57B1/6 mice. Into this pocket, a 0.34 mm×0.34 mm sucrose aluminum sulfate pellet coated with hydron polymer containing 160 ng of recombinant human PDGF-BB, 160 ng of human VEGF-A, or 80 ng of human bFGF was implanted and mice were monitored daily. While those mice implanted with control pellets showed no evidence of angiogenesis, all mice receiving PDGF-BB coated pellets showed evidence of potent angiogenesis (see FIG. 3). Thus, recombinant PDGF-BB protein can potently stimulate the growth of new vessels in both the mouse Matrigel and corneal assays. In contrast to the results obtained using the Matrigel assay, PDGF-BB by itself, although inducing a clear and potent angiogenic response, was less potent than either VEGF-A or bFGF (FIG. 3). The differences observed for the Matrigel and mouse corneas models could be explained by the production of additional endogenous angiogenic factors by transduced myoblasts that synergize more readily with PDGF-BB compared to VEGF-A. Alternatively, the levels of recombinant VEGF-A and PDGF-BB produced in vivo may differ to the levels produced in vitro.

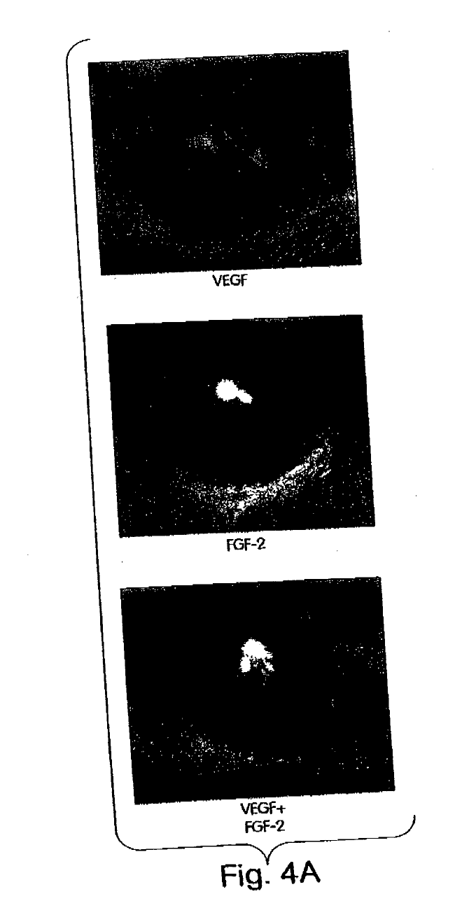

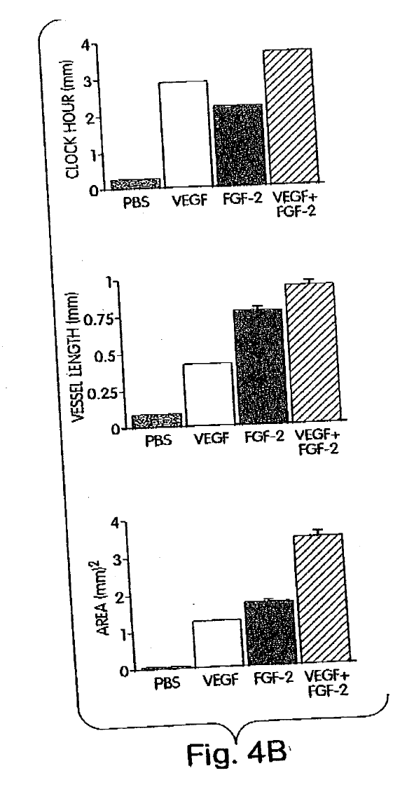

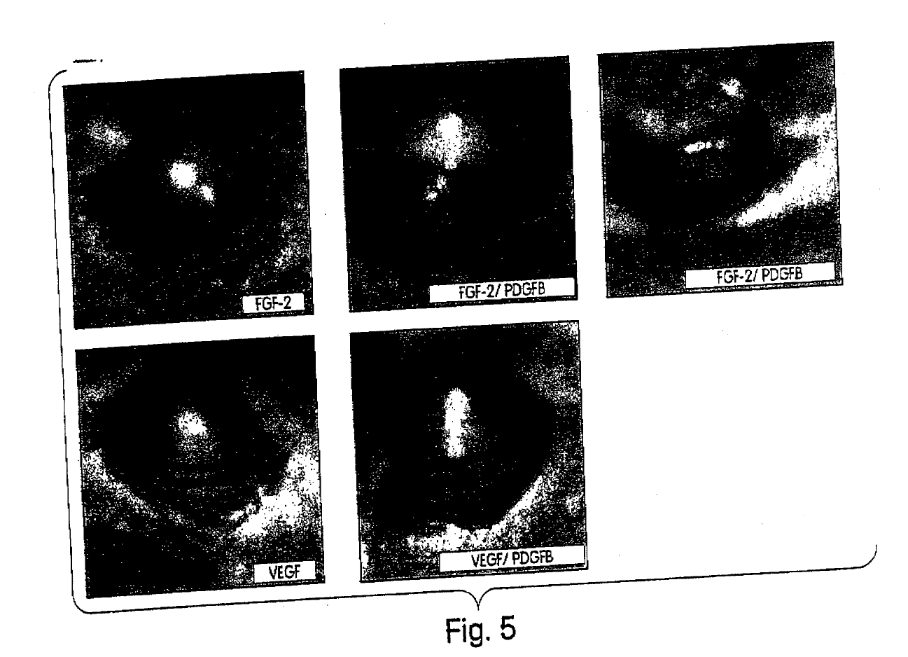

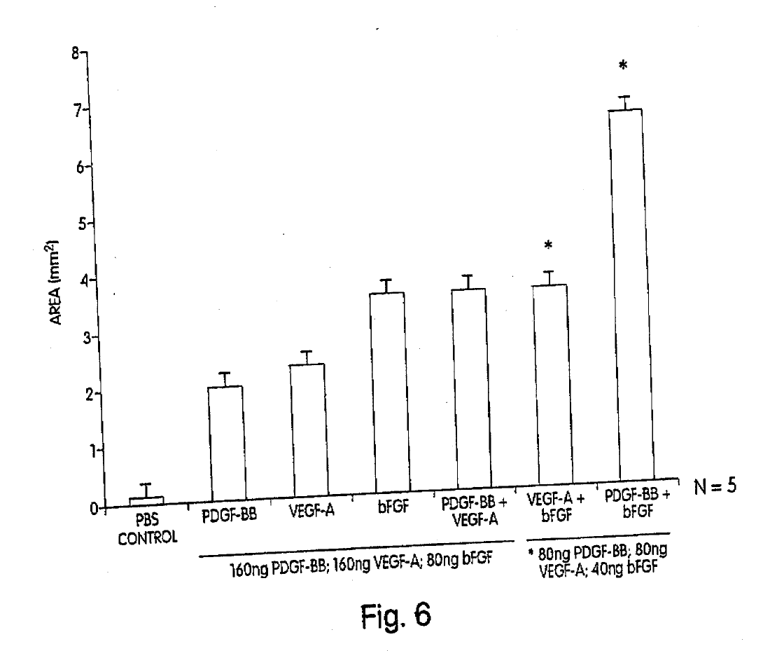

Unexpectedly, the combination of bFGF and PDGF-BB proved to be, by far, the most potent combination (FIG. 5), producing an angiogenic effect many times greater than the any factor alone (FIG. 3) or bFGF and VEGF-A combined (FIG. 4). Moreover, the level of this synergistic effect appeared to be specific to PDGF-BB and bFGF since PDGF-BB combined with VEGF-A did not elicit nearly as potent an effect (see FIG. 6). Therefore, the most potent combination of angiogenic factors observed was PDGF-BB and bFGF.

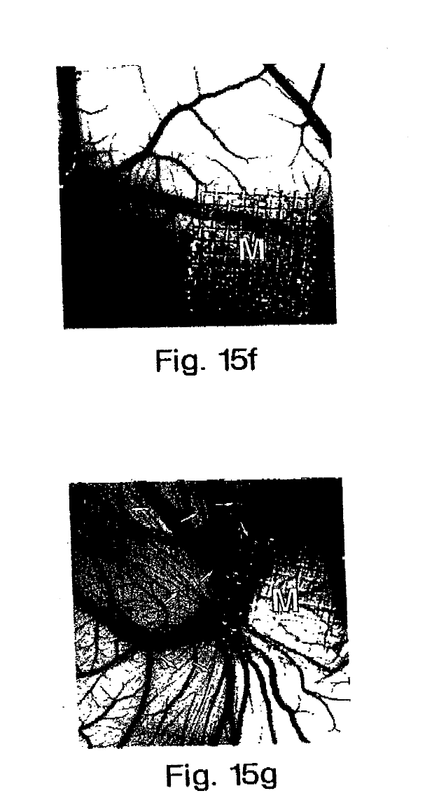

To further study the angiogenic properties of PDGF family members in vivo, three in vivo angiogenesis assays were used for evaluation, namely the mouse corneal micropocket assay, the CAM assay and the mouse Matrigel assay. For the mouse corneal micropocket assay, micropellets containing PDGF-AA, PDGF-AB, or PDGF-BB were surgically implanted into corneas of C57BL/6 mice. Stimulation of new blood vessel growth was examined on day 5 after implantation. All three dimeric isoforms of PDGFs, PDGF-AA, PDGF-AB, and PDGF-BB, were able to induce angiogenesis in the mouse cornea. The angiogenic responses of corneas stimulated by 160 ng of PDGF-AB and PDGF-BB were robust with a high number of capillaries (FIGS. 15c and d). The newly formed vessels, as well as the limbal vessels, were markedly dilated in the PDGF-AB- and PDGF-BB-implanted corneas. The areas of neovascularization stimulated by equivalent amounts of these two factors were indistinguishable from each other (FIG. 15e). In contrast, vessel lengths, vessel clock hours, and vascular areas stimulated by PDGF-AA were significantly less than those induced by PDGF-AB or PDGF-BB (FIGS. 15b and e). Pellets without growth factors serving as negative controls did not induce corneal neovascularization (FIGS. 15a and e).

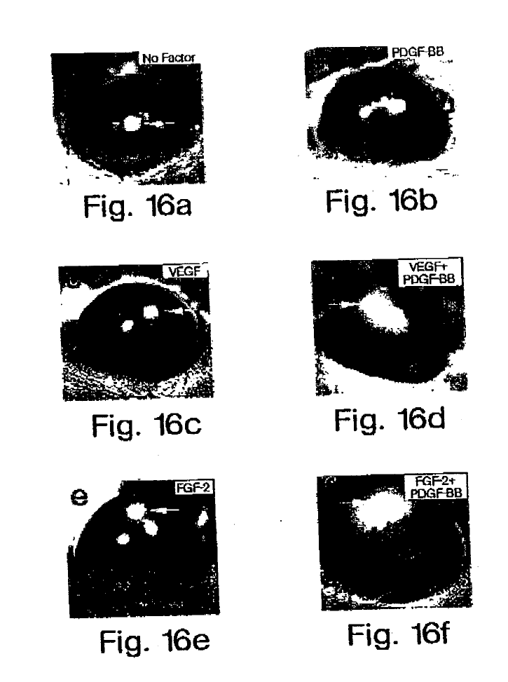

In the CAM assay, PDGF-BB was able to stimulate microvessel growth in each implanted CAM at the dose of 2.5 μg/disc, as seen in FIG. 15g. A significant increase of neovascularization with a high microvessel density was observed in the areas surrounding the PDGF-BB implant (FIG. 15g, arrows). In contrast, discs without growth factors did not stimulate neovascularization in chick embryos (FIG. 15f). In the Matrigel assay, PDGF-BB released from heparin-Sepharose significantly induced neovascularization, shown in FIG. 16i.

### Example 4

### In vivo Assessment of Angiogenic Synergy Between PDGF-BB and bFGF

As described above, PDGF-BB displayed a robust angiogenic response in both the mouse corneal and the CAM models. The following study was performed to investigate the activity of PDGF-BB in combination with other angiogenic factors. Specifically, low amounts of PDGF-BB were co-implanted with bFGF (also referred to as FGF-2) or VEGF into mouse corneas. PDGF-BB alone significantly stimulated corneal angiogenesis as measured by vascular area (FIGS. 16b, g and h). VEGF or FGF-2 induced a more intense corneal neovascularization (FIGS. 16c and e).

Surprisingly, a remarkable synergistic effect on stimulation of corneal blood vessel growth was observed when PDGF-BB was implanted together with FGF-2 (FIGS. 16f and h). The area of neovascularization was significantly increased in PDGF-BB and FGF-2- co-implanted corneas (FIGS. 16f and h) as compared with those stimulated by PDGF-BB (FIG. 16b) or FGF-2 alone (FIG. 16e). The measured neovascularization induced by PDGF-BB and FGF-2 in combination was statistically greater than the sum of the effects obtained with either of these two factors used alone. These newly formed blood vessels were well organized with distinct vascular tree structures and branch formations. In addition to increasing vessel length and area, PDGF-BB/FGF-2 remarkably induced vascular dilation of the newly formed blood vessels (FIG. 16f). Thus, PDGF-BB synergistically stimulated angiogenesis with FGF-2.

In contrast to PDGF-BB- or FGF-2-induced vascular networks, VEGF-stimulated capillary networks appeared as disorganized primitive vascular plexuses with sinusoidal blobs at the leading edges (FIG. 16c). These primitive vascular networks represented the vascular permeability feature induced by VEGF. Combination of PDGF-BB with VEGF did not result in a synergistic effect in promotion of blood vessel growth, other than slightly additive angiogenesis (FIGS. 16d and g). PDGF-BB did not improve the quality of the VEGF-induced vascular networks, which were leaky, tortuous, and primitive vascular plexuses. In contrast, these premature vessels were more leaky than the vessels induced by VEGF alone, with hemorrhages and fusion of capillaries into microvessel blobs (FIG. 16d).

Synergy between PDGF-BB and FGF-2 was also assessed using slow release heparin-sepharose beads. Heparin-sepharose beads loaded with PDGF-BB and/or FGF-2 were subcutaneously injected into the abdominal region of C57BL/6 mice. Fourteen days post-injection, the mice were sacrificed, and pellets removed and sectioned. The number of microvessels was then quantified by visual inspection of the sections under a microscope. PDGF-BB or FGF-2 alone stimulated a 2-fold increase in the number of microvessels observed as compared to the heparin-sepharose control sample, as shown in FIG. 16i. In contrast, the number of microvessels induced by FGF-2/PDGF-BB in combination was greater than 8-fold over control levels (FIG. 16i). Taken together, these data demonstrate that FGF-2/PDGF-BB in combination produces a synergistic efficacy in induction of neovascularization in two in vivo model systems.

### Example 5

### Analysis of how PDGF-BB and bFGF Promote Vascular Stability

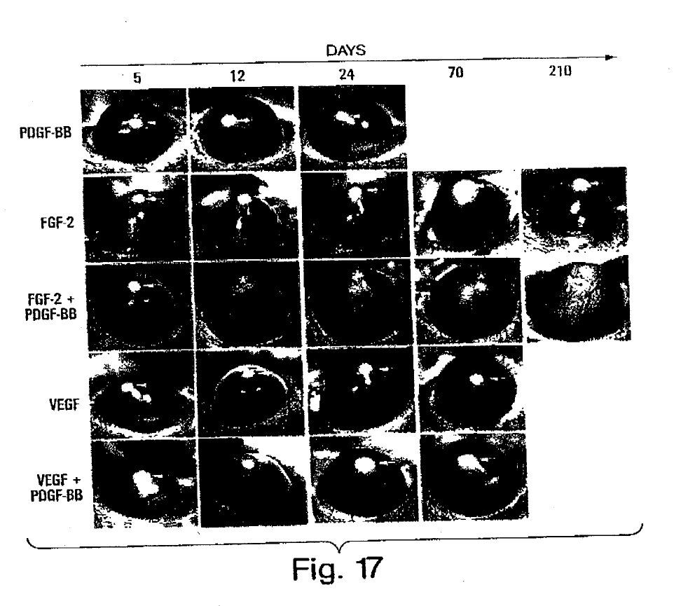

The following example demonstrates that the combination of PDGF-BB and bFGF leads to increased vascular stability. To study the vascular remodeling function of each single growth factor as well as various combinations, induced mouse corneal vascular networks were followed for more than 200 days. Although PDGF-BB induced a robust angiogenic response in corneas between days 5-12 after growth factor implantation (FIG. 17), these vessels completely vanished by day 24. In contrast to

PDGF-BB, FGF-2-induced blood vessels remained relatively stable for 40 days after implantation (FIG. 17), but were virtually all regressed by day 70. Overwhelming corneal neovascularization in the FGF-2/PDGF-BB-implanted corneas between days 7-10 resulted in the loss of all implanted pellets due to a high surface pressure increased by hyperneovascularization. As shown in FIG. 17, corneal neovascularization continued to reach a maximal level at day 12 despite the loss of FGF-2/PDGF-BB implants, and these vascular networks remained stable for over 200 days. These vessels remained stable for more one year without further regression.

In addition to vascular stability, the FGF-2/PDGF-BB-induced vascular networks were remodeled into defined vascular trees (FIG. 17). Similar to FGF-2 alone, the VEGF-stimulated vascular network completely regressed within 70 days after growth factor implantation (FIG. 17), although the initial nascent vascular network with sinusoidal blob-like structures underwent remarkable remodeling to become well-defined tree networks at day 24 (FIG. 17). A combination of PDGF-BB/VEGF significantly improved the remodeling and stability of blood vessels (FIG. 17) compared to either factor used alone, however, after a prolonged period of about 70 days, the number of vessels found was barely detectable. These data demonstrate that PDGF-BB stabilized FGF-2- and slightly stabilized VEGF-induced vascular networks.

### Example 6

### Further Assessment of the Effects of PDGF-BB and bFGF

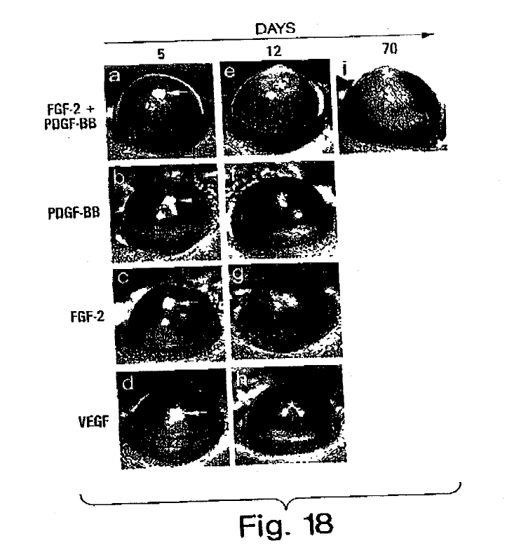

The following example demonstrates that exposure to FGF-2 in combination with PDGF-BB leads to the establishment of stable corneal vascular networks. This example further reveals that this establishment is not due to differences in the bioavailability of angiogenic factors as they are released from slow release polymers. Implanted growth factor pellets in the corneas were deliberately removed on day 6 after implantation. Nascent vascular networks induced by PDGF-BB (FIG. 18b), FGF-2 (FIG. 18c) or VEGF (FIG. 18d) were all completely regressed by one week after removal of growth factors (FIGS. 118f-h). In contrast, FGF-2/PDGF-BB-induced vascular networks remained for at least 70 days without regression of microvessels (FIGS. 18h and i). These well-established and defined vascular trees remained for more than 200 days without further regression. These data indicated that PDGF-BB, FGF-2 and VEGF acted as survival factors for their own premature and nascent vascular networks. The long-term stability of vascular networks is independent from the resource of growth factors and is determined at an early phase of neovascularization by a transient exposure to a group of angiogenic factors, such as FGF-2 and PDGF-BB in combination. A combination of PDGF-BB and FGF-2 led to the establishment of stable vascular networks, even after the depletion of growth factors.

### Example 7

### Assessment of the Correlation of Mural Cell Association and Corneal Vessel Stability

The following example demonstrates that the combination of FGF-2 and PDGF-BB leads to newly formed vasculature with an increased rate of maturity. To assess the maturity of corneal vessels induced by various growth factors, histological examination was performed by double-staining corneal sections for the presence of CD31, an endothelial cell marker, and desmin, a specific marker expressed in pericytes and smooth muscle cells. Histological sections of FGF-2-, PDGF-BB-, and PDGF-BB/FGF-2-implanted corneas at day 5, day 12 and day 24 were incubated with an anti-CD31 antibody and stained with a FITC-conjugated secondary antibody to visualize positive signals in green. The same corneal sections were stained with a biotinylated antibody against mouse desmin and positive signals were visualized as red. The CD31 positive signals were superimposed with desmin positive signals with a digital imaging program to create double staining signals. Overlapping signals represent microvessels coated with mural cells.

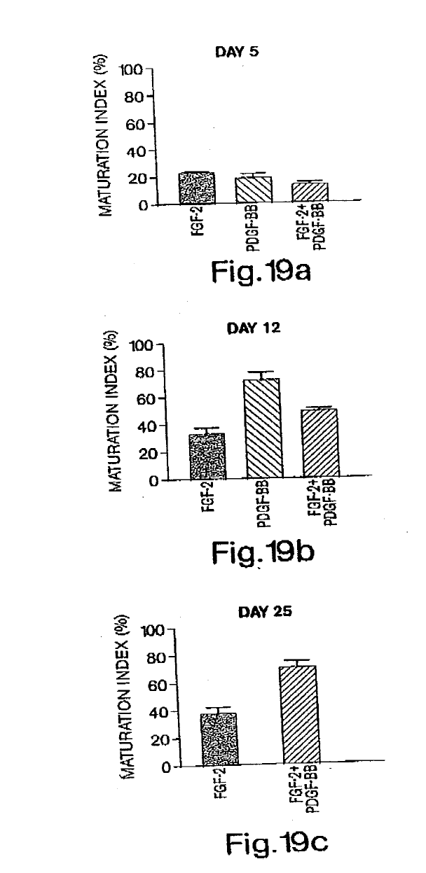

The numbers of corneal microvessels positively stained by desmin were fewer as compared with CD31 positive vessels. Quantification of desmin positive signals revealed no significant difference in FGF-2-, PDGF-BB- or PDGF-BB/FGF-2-induced vessels at day 5. An overlapping pattern was detected with both CD31 and desmin positive signals when images of the same sections were combined digitally. A maturation index (% of vessels coated with periendothelial cells) of about 15-20% was found in corneal vessels induced by FGF-2, PDGF-BB or PDGF-BB/FGF-2 at day 5 and no significant difference in maturity of these various factors-induced blood vessels was recorded, as described in FIG. 19a. At day 12, a significant increase in maturation index to about 60% was found in the PDGF-BB-induced vessels as compared with FGF-2 alone (about 30%) (FIG. 19b). A significant increase of smooth muscle cell coating (about 45%) was also found in the PDGF-BB/FGF-2 co-implanted vessels. At day 25, more than 70% of corneal vessels induced by FGF-2/PDGF-BB were desmin positive, whereas less than 40% of FGF-induced vessels were associated with mural cells (FIG. 19c). By this time, all PDGF-BB-induced vessels had regressed. These data demonstrate that microvessel stability requires a simultaneous exposure of FGF-2 and PDGF-BB to the newly formed vascular network and correlates with increase of vascular maturation.

### Example 8

### Analysis of Stimulation of Collateral Growth and Improvement of Blood Perfusion in Ischemic Hind Limbs

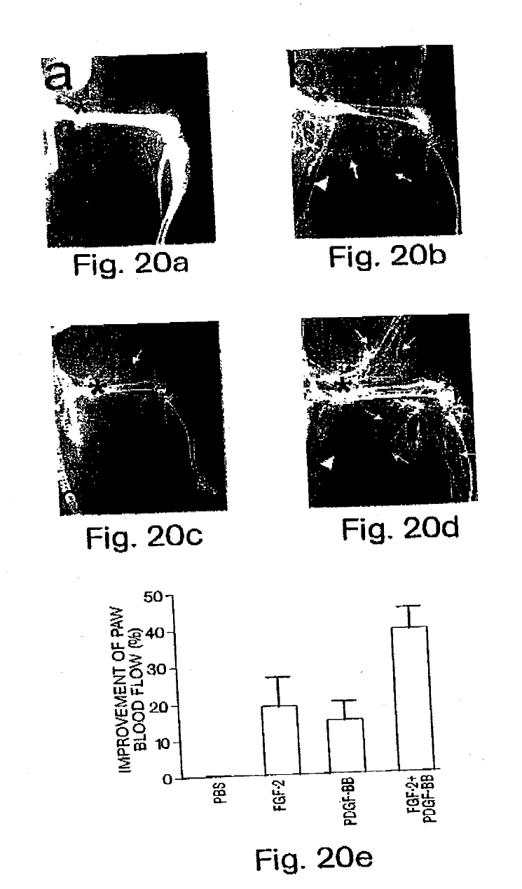

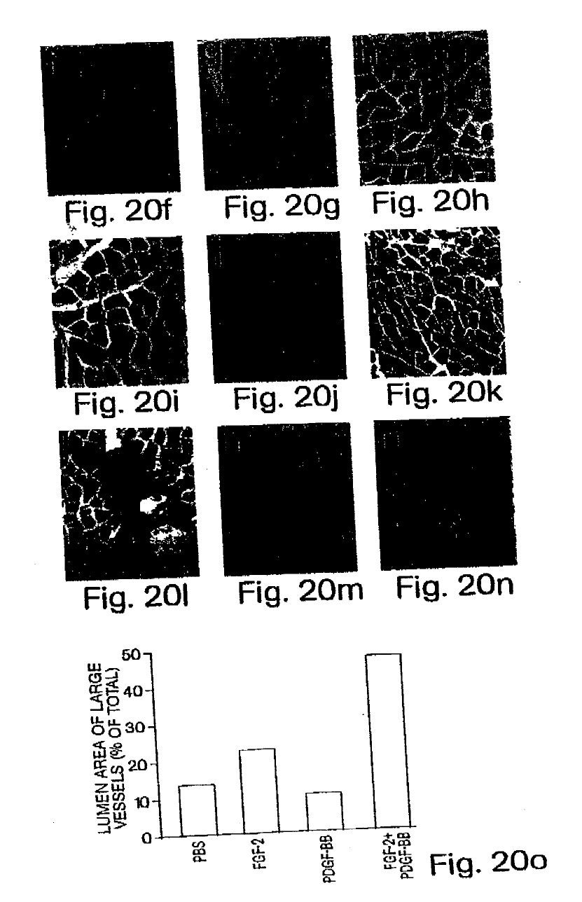

The following example is provided to show that FGF-2 and PDGF-BB together in combination can promote angiogenesis in ischemic tissues. An ischemic hind limb model for therapeutic angiogenesis was used to test whether FGF-2 and PDGF-BB in combination could induce collateral arteriogenesis in ischemic tissues. Angiographic analysis at day 23 after ligation of femoral artery showed that a number of collateral vessels were found in the FGF-2-treated group (FIG. 20b). Interestingly, delivery of PDGF-BB in ischemic muscles produced only a moderate effect in stimulating collateral growth (FIG. 20c). In contrast, FGF-2/PDGF-BB in combination stimulated collateral growth and resulted in high density of collaterals, which were distributed in broad areas of ischemic muscles found near the lesion site and extended to the distal regions of the ischemic limb (FIG. 20d, arrows). These newly formed collaterals were highly dilated as compared with those induced by FGF-2 alone (FIGS. 20d and 20b, arrowheads). As a negative control, PBS buffer alone did not significantly induce collateral growth (FIG. 20a).

Because angiographic analysis was aimed to detect relatively large collateral vessels, immunohistochemical analysis was performed using an anti-α-SMA antibody to investigate if these collateral vessels were different among various groups. In consistency with the angiographic analysis, dual angiogenic factor delivery resulted in a dramatic increase of relatively large arterial vessels (FIGS. 20l-n). The lumen area of these large arterial vessels (>700 μm²) occupied nearly 50% of the total lumen area in histological sections of the dual angiogenic factor-treated group (FIG. 20o). In contrast, FGF-2- (FIGS. 20h and i) and PDGF-BB- (FIGS. 20j and k) stimulated collaterals mainly consisted of smaller arterial vessels with an average lumen area of majority of vessels<400 μm^{2.} Quantification analysis revealed that large arterial vessels (>700 μm²) in the FGF-2-treated and PDGF-BB-treated groups occupy only about 24% and 10%, respectively (FIG. 20o). Delivery of PBS buffer alone did not result in a significant increase of the number of large arteries (FIG. 20f, g and o).

In agreement with the increase of lumen areas in the dual angiogenic factor delivery group, paw blood perfusion in the ischemic hind limbs was dramatically improved with an average increase of about 40% above the PBS buffer-treated group (FIG. 20e). Delivery of FGF-2 or PDGF-BB alone improved the blood perfusion by 15-20%, as compared with the PBS treated group (FIG. 20e). These data demonstrate that dual angiogenic factor delivery induces functional collaterals and that the combination of FGF-2 and PDGF-BB resulted in dramatic stimulation of collateral growth and improved blood perfusion in ischemic rat hindlimbs.

### Example 9

### Analysis of the Upregulation of PDGF Receptors by FGF-2

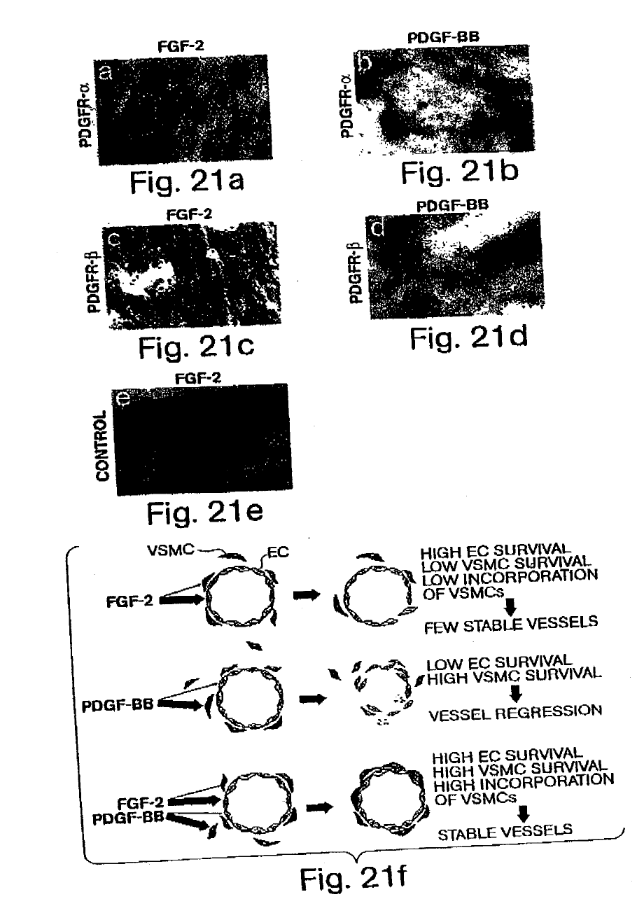

To further study the observed synergistic effects of FGF-2 and PDGF-BB on angiogenesis and vessel stability/remodeling, localization patterns of PDGF receptors, PDGFR-α and -β, were examined in the newly formed corneal blood vessels. Expression of PDGFR-α and -β mRNAs was detected by in situ hybridization using probes for each of these receptors (Yarden, et al. (1986) Nature 323, 226-32). Both PDGFR-α and -β were expressed in PDGF-BB-induced microvessels (FIGS. 21a and b). However, significantly higher levels of these PDGF receptors were detected in FGF-2-induced vessels (FIGS. 21c and d). Both PDGFR-α and -β positive signals were distributed in newly formed blood vessels, as confirmed by co-localization of anti-CD31 staining. These data further demonstrate that angiogenic synergy requires that FGF-2 upregulates the expression levels of PDGF receptors, which transduce angiogenic and arteriogenic signals triggered by PDGF-BB.

The results shown in FIG. 21 demonstrate that FGF-2 is a potent angiogenic factor that preferentially acts on endothelial cells (EC) in vivo. FGF-2 is also a key survival factor for endothelial cells. However, FGF-2 had only poor survival effect on vascular smooth muscle cells (VSMC). Thus, FGF-2-induced vessels were not stable. PDGF-BB was a potent mitogenic and chemotactic factor for VSMC. However, it was a poor survival factor for EC. As a result, PDGF-BB-induced vascular networks regress. FGF-2/PDGF-BB in a combination produced not only synergistic effect on angiogenesis, but also confered potent survival effect on both EC and VSMC. Thus, these vessels remained stable.

### Example 10

### In vivo Analysis of PDGF-BB on Heart Tissue Remodeling

To study the effects of PDGF-BB on chronic myocardial ischemia, pigs were used as the model system. PDGF-BB was applied intramyocardially through a slow release formulation to ischemic pigs with induced chronic myocardial. Pigs were then observed and monitored for myocardial blood flow and general cardiac function both at rest and during stress. Results from the experiment showed no side effects in the pigs injected with high doses of PDGF-BB.

Results from the experiment showed that PDGF-BB dose dependently increased collateralization, regional myocardial blood flow, and improved regional wall motion. The results were more striking under stress conditions than the results obtained during rest conditions due to better standardized hemodynamic conditions during stress than at rest, and by the exaggeration of flow and function differences under stress conditions.

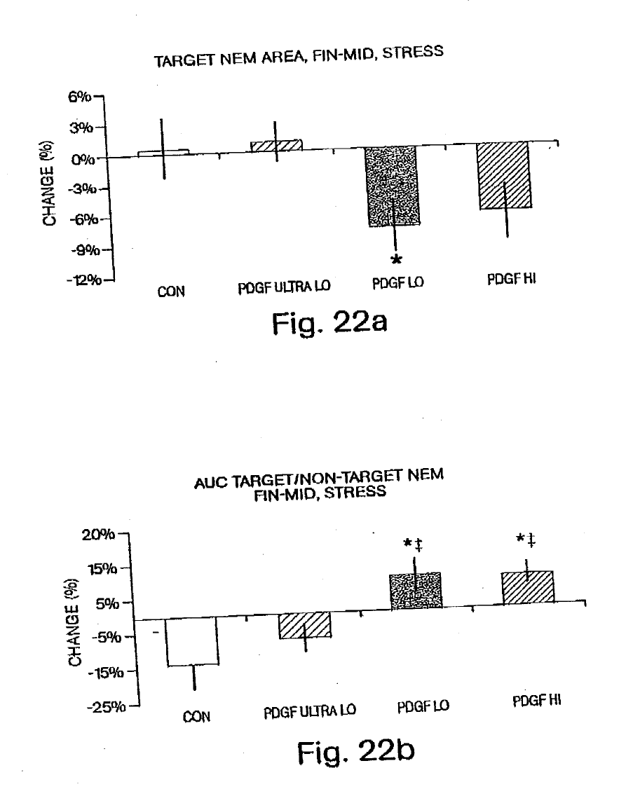

Normalized endocardial motion (NEM) and normalized wall thickening (NWT) were analyzed separately. Results from the stress data are shown in FIG. 22. The extent of NEM deficit at stress did not change in the control and ultralow dose PDGF-BB group (FIG. 22A), and reduced in the low and high dose groups (ANOVA p=0.05), with the largest differences between the low dose and the control and ultralow dose group (p=0.03 for both). As shown in FIG. 22A, the PDGF lo and hi group improved (i.e. reduced area of motion defect), as the lo group was significantly different from the control and the ultra lo group (P<0.05). A similar result was obtained when the ratio of AUCtarget/AUCnon-target was used as measure of regional wall motion (FIG. 22b). As shown in FIG. 22b, PDGF lo and PDGF hi were significantly different from control (p<0.01) and from PDGF ultra lo (p<0.04). No effect was observed on normalized wall thickening, regardless of the parameter that was chosen (ANOVA p-values ranging from 0.76 to 0.98). The latter result was consistent with results obtained from the M-mode echocardiography.

Results from histological analysis of pig myocardial tissue showed that there were no signs of infiltration or uncontrolled angiogenesis which could have resulted from high doses of PDGF-BB. No changes were observed in target wall thickening, using M-mode echocardiography and regional tracking of wall thickening over the entire circumference of the heart. This parameter was less sensitive than wall motion since the epicardial lining is more difficult to trace and small tracing errors result in big changes in wall thickening. Collectively, these results demonstrate that PDGF-BB improved remodeling of the normal ventricle and thereby improved regional function, for instance in fractional shortening.

### Example 11

### Therapeutic Effect of PDGF-BB Delivered Via Slow Release Microcapsules for the Treatment of Myocardial Ischemia

The following studies were performed to assess the ability of PDGF-BB to promote angiogenesis in vivo in the stringent Matrigel model and to improve cardiac function in ischemic rat models having myocardial infarction, using a slow release delivery system employing heparin-sepharose/alginate microcapsules. This delivery approach was based upon the ability of certain factors, such as bFGF and PDGF-BB, to bind strongly to heparin molecules both in vitro and in vivo.

Sepharose beads coated with heparin (approximately 50-150 μm in size) were purchased from Pharmacia. The beads were sterilized using UV irradiation and mixed with a 1.6% solution of alginate polymer. This polymer is able to form gels through chemical cross-linking with multivalent cations such as calcium. The procedure for making heparin-sepharose/alginate capsules, shown in FIG. 7, was as follows. Sterilized heparin-sepharose beads were mixed with a 1.6% alginate solution and the mixture was loaded into a 5 ml syringe. The mixture was then extruded through a needle and a mist of heparin sepharose/alginate, produced using a coaxial air flow system, dropped into a wash bath of 1.5% calcium chloride solution. Once the alginate hit the calcium solution, the alginate became cross-linked, forming a solid gel capsule in the shape of a sphere. Once formed, the capsules were forced through a 250 μm sieve, washed twice in sterile water and stored in buffer composed of 0.9% sodium chloride and 1 mM calcium chloride. Visual analysis of the capsules under the microscope showed that the vast majority of microcapsules were composed of individual heparin sepharose beads coated with a thin layer of alginate.

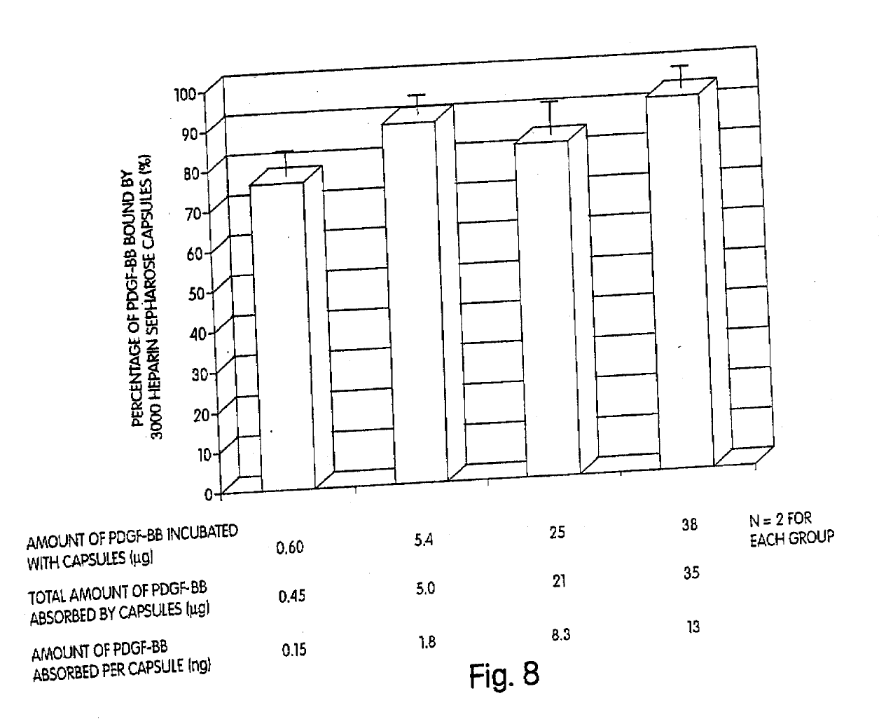

Heparin-sepharose/alginate microcapsules were incubated overnight at 4 degrees Celcius in binding buffer composed of 0.9% sodium chloride, 1 mM calcium chloride, 0.05% gelatin and 10 μg of recombinant PDGF-BB for 16 hours. The next day, the binding buffer was removed from the microcapsules and analyzed by ELISA to quantify the amount of PDGF-BB absorbed by the capsules. In a typical experiment, approximately 75-90% of the PDGF-BB protein is absorbed by the microcapsules (see FIG. 8). Next, the heparin-sepharose/alginate microcapsules were washed twice in fresh binding buffer and either placed in vitro to assess release kinetics or injected into the myocardium of rats that had undergone surgically induced myocardial infarction.

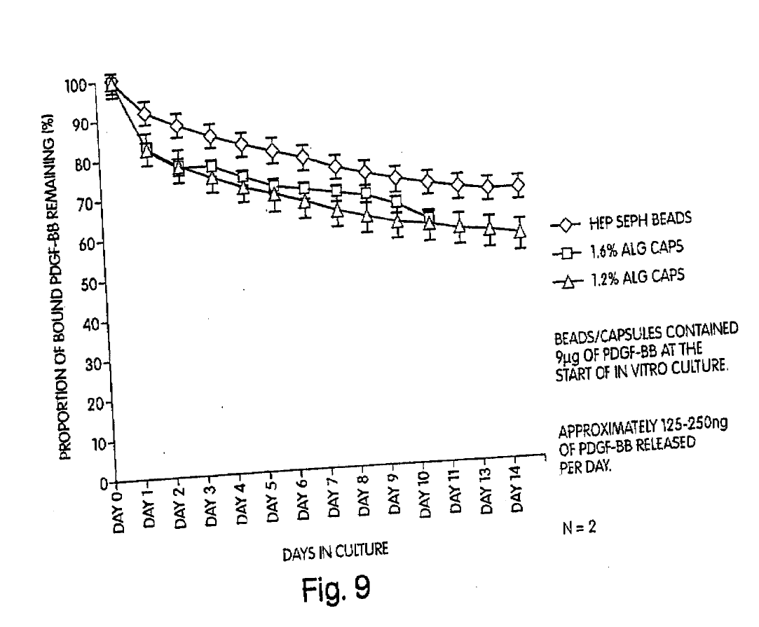

To assess the release kinetics of the bound PDGF-BB in vitro, three thousand heparin-sepharose/alginate microcapsules or three thousand non-alginate encapsulated heparin sepharose beads were incubated with PDGF-BB at 4° C. overnight with gentle shaking. ELISA analysis of the binding buffer the next day showed absorption of 90% (9 μg) of the PDGF by the capsules. Following incubation with PDGF-BB, the beads/microcapsules were washed, resuspended in 5 mls of serum free medium and incubated at 37° C. Every 24 hours the medium was changed and the amount of PDGF-BB present in the medium quantified by ELISA. The results showed a slow, sustained release of approximately 0.5-3% of the total bound PDGF-BB (representing 125-250 ng) which was detected each day for a minimum of 14 days, the longest time point tested (see FIG. 9). Importantly, the proportion of PDGF-BB released per day was equivalent to the amount of PDGF-BB that was estimated to be secreted by muscle cells transduced with the PDGF-BB retrovirus in the Matrigel experiments described in Example 1. As shown in FIG. 9, the release kinetics for non-encapsulated heparin sepharose beads was better than those observed for the alginate encapsulated heparin sepharose.

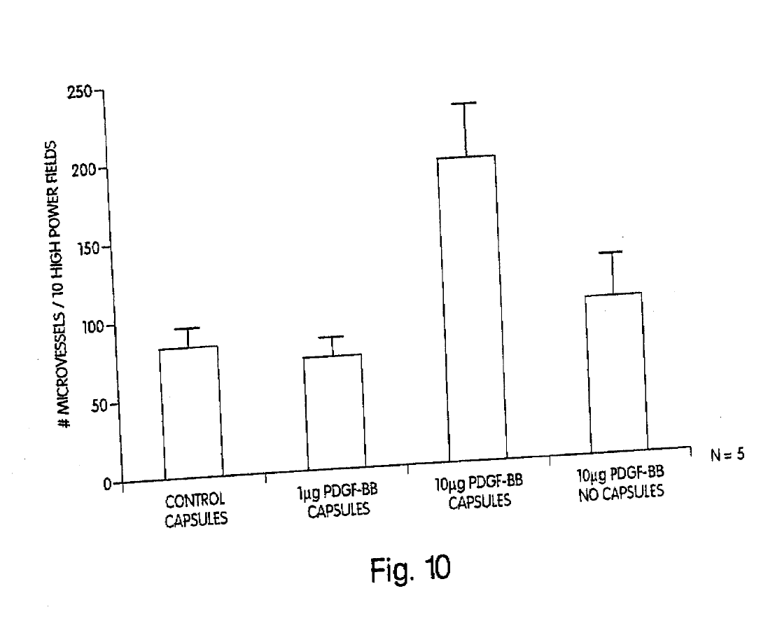

The ability of the heparin-sepharose/alginate microcapsules to stimulate angiogenesis in vivo was assessed using the stringent Matrigel assay. Three thousand microcapsules loaded with 1 μg or 10 μg of PDGF-BB were mixed with 400 μl of Matrigel and subcutaneously injected into the abdominal region of C57B1/10 mice. Thirteen days later mice were sacrificed, the pellets and a section of the adjacent abdominal muscle was removed, fixed, sectioned and the number of microvessels quantified by visual inspection of the sections under the microscope. As shown in FIG. 10, the number of microvessels in mice receiving microcapsules loaded with 10 mg of PDGF-BB was 2.5-fold greater than that of control mice.

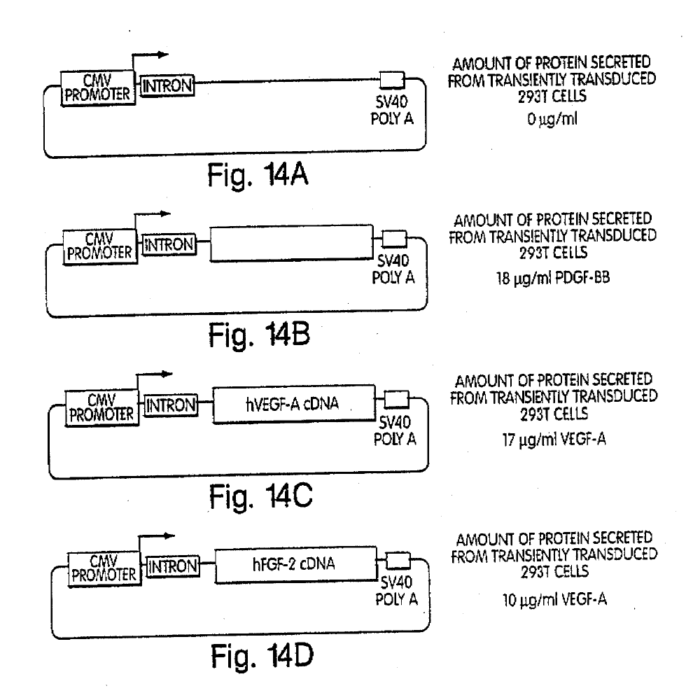

In addition, as shown in FIG. 13, PDGF-BB and bFGF delivered by slow release microcapsules synergize to stimulate angiogenesis in vivo in the stringent Matrigel model. Three thousand microcapsules loaded with 1 μg of bFGF were mixed with 400 μl of Matrigel and subcutaneously injected into the abdominal region of C57B1/10 mice. Thirteen days later mice were sacrificed, the pellets and a section of the adjacent abdominal muscle was removed, fixed, sectioned and the number of microvessels quantified by visual inspection of the sections under the microscope. FIG. 13 shows that the number of microvessels in mice receiving bFGF+PDGF-BB microcapsules was 4-fold greater than that of mice implanted with either growth factor alone.

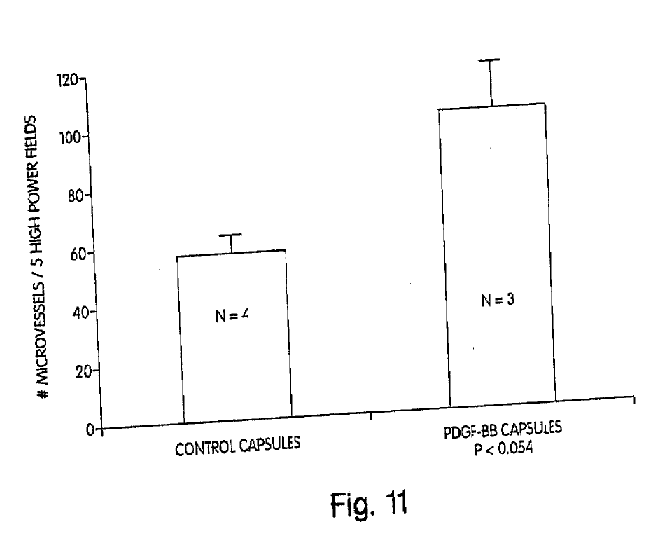

PDGF-BB microcapsules were also tested for their ability to stimulate angiogenesis in infarcted rat hearts 3 weeks post-injection. Infarcted rat hearts were injected with 1600 microcapsules containing μg (control) or 18 μg of PDGF-BB in a volume of 20 μl. Three weeks post injection rats were sacrificed, hearts were removed, fixed, sectioned and the number of microvessels within the infarct region quantified by visual inspection under a microscope (i.e., number of microvessels per 5 high power fields for recipients of control and PDGF-BB microcapsules). As shown in FIG. 11, rats injected with PDGF-BB microvessels showed an approximate 2-fold increase in the number of microvessels as compared to control rats.

The ability of the heparin-sepharose/alginate microcapsules to stimulate angiogenesis in vivo was also assessed using the ischemic rat heart model as follows. Adult male rats were anesthetized, intubated and ventilated with a Harvard respirator. Under sterile conditions, a left lateral thoractomy was performed. The heart was exposed and the left descending coronary artery was ligated with a 8-0 Prolene suture. Immediately after infarction each heart was injected twice intramyocaridally with 10 μl of a buffer suspension containing approximately 800 heparin sepharose/alginate microcapsules with 9 μg of absorbed recombinant human PDGF-BB protein. Thus, a total of 1600 microcapsules containing 18 μg of human PDGF-BB protein were injected into each rat heart. The lungs were then inflated and the wound was closed in layers.

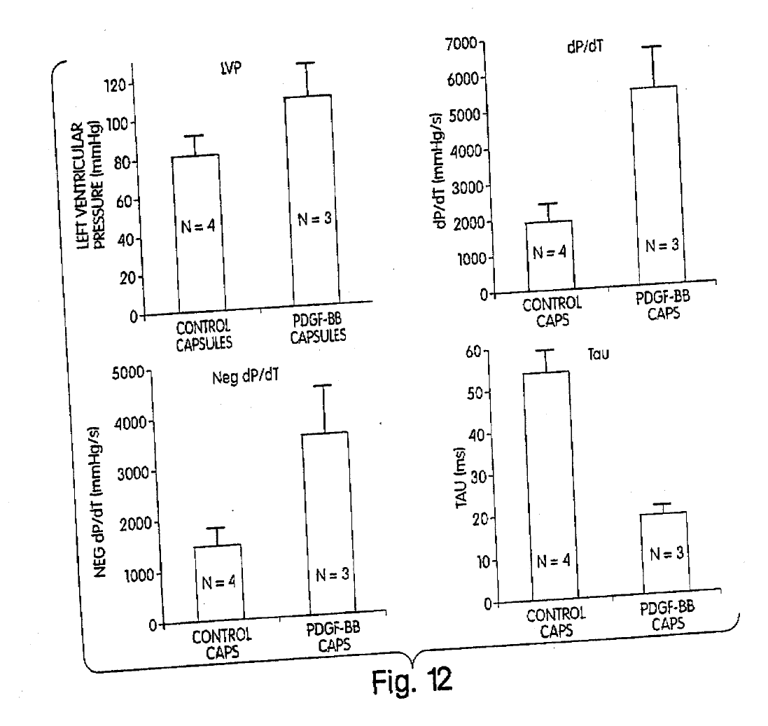

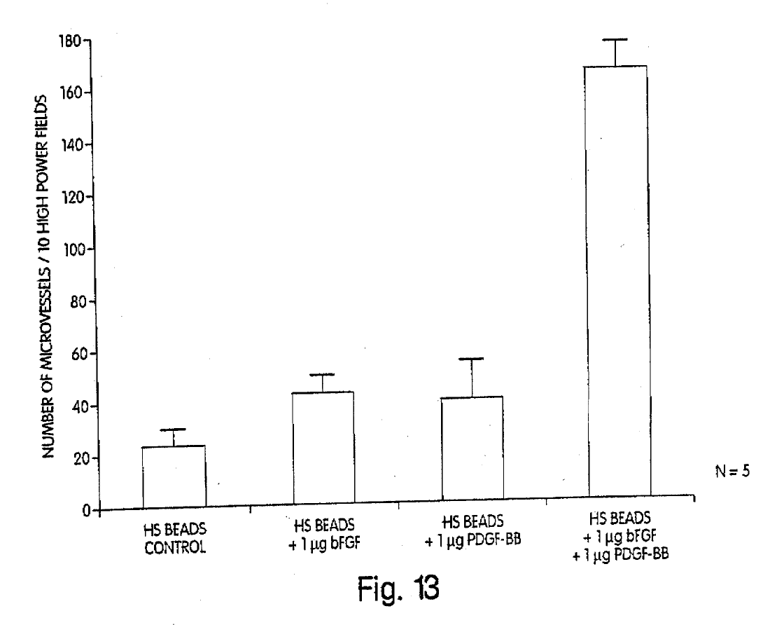

Three weeks later, cardiac function was assessed using a variety of parameters including left ventricular pressure (LVP), dP/dT (a measure of cardiac contractility), negative dP/dT (a measure of relaxation of the cardiac muscle) and tau (the relaxation constant) (see FIG. 12). Rats injected with PDGF-BB microcapsules, showed a 25% increase in left ventricular pressure (see FIG. 12). Moreover, cardiac contractility/relaxation increased 2-3-fold while the relaxation constant, tau, was decreased by 2.5-3-fold (FIG. 12). Thus, a significant improvement in all parameters was detected in rats injected with PDGF-BB microcapsules.

### Example 12

### Directed Delivery of Angiogenic Factors Using Biocompatible Threads

Regulating the growth of new vessels from existing vasculature (angiogenesis) that effectively bypass an arterial lesion requires strict spatial and temporal control. Angiogenic factors, such as those described in the preceding examples, work by providing a gradient of angiogenic factor that stimulates the chemotaxis and proliferation of endothelial cells, and their supporting cells towards the source of the factor.

Today, most efforts to stimulate the growth of new vessels involve the injection of proteins into or near affected areas. Such injections can result in the spread of the angiogenic factor over a large area, greatly diminishing their efficacy, and can dilute the angiogenic stimulus over a large region resulting in an unorganized hodge-podge of new vessels that do not provide any therapeutic benefit.

To solve this problem, biocompatible absorbable threads containing angiogenic factors can be employed to provide a small, highly localized and orderly gradient of angiogenic factors in the appropriate and crucial areas. This enables the creation of a “molecular road map” that directs the growth of new vessels from around the site of the arterial occlusion to join again at a point below or downstream of the blockage. To achieve this, absorbable surgical threads can be coated with the appropriate angiogenic factors (e.g., PDGF-BB, FGF-2, VEGF-A and PDGF-B). Such threads can then be surgically placed at the site of arterial occlusion such that they provide a clear spatial direction and gradient of angiogenic factor(s) to direct the generation of new vessels around the block.

The biocompatible threads can be coated with one or several angiogenic factors to allow delivery of growth factor or growth factor combinations that provide the optimal angiogenic stimulus. Moreover, such threads can be engineered to release certain growth factors at specific rates and at specific times that may help mimic the natural angiogenic process more closely.

### Example 13

### Treatment of Ischemia Using Angiogenic Expression Plasmids

FIG. 14 shows a variety of expression plasmids encoding angiogenic factors that can be administered directly to localized areas of tissue to promote angiogenesis.

To show that the plasmids encode biologically active PDGF-BB protein, supernatant from 293T cells transiently transfected with the PDGF-BB expression plasmid was added to NIH 3T3 cells growing under serum free conditions. Seventy two hours later cells were trypsinized, spun down and counted using a hemocytometer. The results showed that the PDGF-BB supernatant specifically and potently induced the proliferation of NIH3T3 cells.

To analyze cardiac function following administration of expression plasmids in vivo, test animals (e.g., rats) can be injected with control vs. angiogenic plasmids (e.g., PDGF-BB expression plasmids) following myocardial infarction. To achieve this, test animals can be anesthetized and intubated. The chest wall is opened and a myocardial infarct is created by tying off the anterior descending artery. 180 μg of control or test expression plasmid is injected into the heart wall in a volume of 20 μl. Cardiac function is assessed 3 weeks post injection. Animals are sacrificed, the heart is removed and efficiency of plasmid uptake is assessed by staining with X-gal. The size of the infarct and the extent of angiogenesis is quantified.

Incorporation by Reference

The contents of all references and patents cited herein are hereby incorporated by reference in their entirety.

Equivalents

Although the invention has been described with reference to its preferred embodiments, other embodiments can achieve the same results. Those skilled in the art will recognize or be able to ascertain using no more than routine experimentation, numerous equivalents to the specific embodiments described herein. Such equivalents are considered to be within the scope of this invention and are encompassed by the following claims.

What is claimed is:

1. A method of treating tissue ischemia comprising delivering a combination of PDGF-BB and bFGF to an area of tissue in an amount effective to treat the ischemia by forming vessels that remain stable after the delivered PDGF-BB and bFGF are no longer present.

2. The method of claim 1, wherein the tissue ischemia is myocardial ischemia.

3. The method of claim 1, wherein the combination of PDGF-BB and bFGF further promotes tissue remodeling.

4. The method of claim 1, wherein the combination of PDGF-BB and bFGF is delivered via a NOGA delivery system.

5. The method of claim 1, wherein the tissue ischemia comprises one or a combination of pulmonary ischemia, limb ischemia, brain ischemia, retinal ischemia, nerve tissue ischemia, kidney ischemia, skin ischemia, subcutaneous tissue ischemia, ischemia of the gut and ischemia of the brain.

6. The method of claim 1, wherein PDGF-BB and bFGF are delivered in further combination with one or more other angiogenic proteins.

7. The method of claim 6, wherein the angiogenic protein is selected from the group consisting of PDGF-AA, M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof.

8. The method of claim 1, wherein the combination of PDGF-BB and bFGF is administered locally in the form of a protein composition.

9. The method of claim 8, wherein the combination of PDGF-BB and bFGF is delivered in the form of a sustained release composition.

10. The method of claim 9, wherein the combination of PDGF-BB and bFGF is delivered in association with a polymer.

11. The method of claim 10, wherein the polymer comprises a matrix.

12. The method of claim 11, wherein the matrix is selected from the group consisting of heparin sepharose/alginate, chitosan/tricalcium phosphate sponge, poly-lactide-glycolide sponge, polylactide glycolic mesh, methyl cellulose, polysulfone, extrudable ethylene vinyl acetate, alginate/poly-L-lysine/alginate and agarose/poly-L-lysine/alginate.

13. The method of claim 1, wherein the combination of PDGF-BB and bFGF is delivered by expression from DNA encoding the factors.

14. The method of claim 13, wherein the DNA is contained within a vector.

15. The method of claim 14, wherein the DNA is contained within a vector selected from the group consisting of an adenoviral vector, a retroviral vector, an adeno-associated viral vector, an RNA vector, a liposome, a cationic lipid, a lentiviral vector and a transposon.

16. The method of claim 15, wherein the lentiviral vector is selected from the group consisting of HIV, FIV, BIV, EIAV, and SIV.

17. A method of promoting tissue remodeling comprising delivering PDGF-BB to a localized area of tissue in an amount sufficient to cause tissue remodeling.

18. The method of claim 17, wherein PDGF-BB is delivered in further combination with bFGF.

19. The method of claim 17, wherein PDGF-BB is delivered via a NOGA delivery system.

20. The method of claim 17, wherein the tissue comprises ischemic tissue.

21. A method for promoting angiogenesis comprising contacting a localized area of tissue with heparin sepharose-containing microcapsules in an amount effective to induce angiogenesis within the area of tissue.

22. The method of claim 21, wherein the microcapsules comprise heparin sepharose in a form selected from the group consisting of heparin sepharose beads, heparin sepharose beads coated with a single layer of alginate polymer, heparin sepharose coated with poly-ethylene glycol (PEG) polymer, and heparin sepharose beads coated with alternating layers of alginate and PEG.

23. The method of claim 21, wherein the microcapsules range in size from 1-250 microns.

24. The method of claim 22, wherein the heparin sepharose beads encapsulate an angiogenic factor selected from the group consisting of M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, PDGF-BB, PDGF-AA, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof, or an expression vector encoding said angiogenic factor.

25. The method of claim 24, wherein the angiogenic factor is purified from its native source or produced by recombinant expression.

26. The method of claim 21 wherein the microcapsules are contacted with the localized area of tissue by injection or surgical implantation.

27. The method of claim 26 wherein the injection is performed using a catheter based trans-myocardial injection technology (i.e. NOGA).

28. The method of claim 13, wherein the angiogenic factor is control released from the microcapsule into the localized area of tissue.

29. The method of claim 21, wherein the induction of angiogenesis is used to treat ischemia.

30. A method for promoting angiogenesis comprising contacting a localized area of tissue with a gradient of one or more angiogenic factors or a nucleic acid encoding one or more angiogenic factors, such that directed vascular growth along the gradient is achieved.

31. The method of claim 30, wherein the directed vascular growth results in interconnection of blood vessels.

32. The method of claim 30, wherein the directed vascular growth results in intraconnection of blood vessels.

33. The method of claim 30, wherein the directed vascular growth circumvents blood flow around a blockage within a blood vessel.

34. The method of claim 30, wherein the angiogenic factor or nucleic acid is released in a gradient from a biocompatible material contacted with the localized area of tissue.

35. The method of claim 34, wherein the biocompatible material is a polymer or thread which incorporates the angiogenic factor.

36. The method of claim 35, wherein the biocompatible material comprises an absorbable thread.

37. The method of claim 36, wherein the thread comprises a material selected from the group consisting of polyglyconate monofilament, poliglecaprone 25-(Monocryl), polydiaxonone (PDS II), polyglactin 910, polyglycolic acid, Biodyn glycomer 631, chromic surgical gut and plain surgical gut.

38. The method of claim 34, wherein the biocompatible material is implanted into the localized area of tissue.

39. The method of claim 30, wherein the nucleic acid is contained in an adenoviral vector, retroviral vector, adeno-associated viral vector, RNA vector, liposome, cationic lipid, lentiviral vector, AAV or transposon.

40. The method of claim 39, wherein the lentiviral vector is selected from the group consisting of HIV, FIV, BIV, EIAV, and SIV.

41. The method of claim 30, wherein the one or more angiogenic factors is selected from the group consisting of M-CSF, GM-CSF, VEGF-A, VEGF-B, VEGF-C, VEGF-D, VEGF-E, neuropilin, FGF-1, FGF-2(bFGF), FGF-3, FGF-4, FGF-5, FGF-6, PDGF-BB, PDGF-AA, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof.

42. The method of claim 30, wherein the induction of angiogenesis is used to treat ischemia.

43. A method for promoting angiogenesis comprising:
applying one or more angiogenic factors, or a nucleic acid encoding one or more angiogenic factors, to a biocompatible material to form a treated material; and
contacting the treated material with a localized area of tissue, such that the angiogenic factor or nucleic acid is released into the surrounding tissue in a directed gradient.

44. The method of claim 43, wherein the biocompatible material is an absorbable thread.

45. The method of claim 44, wherein the thread is surgically implanted into the localized area of tissue.

46. The method of claim 43, wherein the one or more angiogenic factors includes PDGF-BB.
